(19) United States
(12) Patent Application Publication
(10) Pub. No.: US 2023/0183224 A1
(43) Pub. Date: Jun. 15, 2023

(54) BEZOXAZINE DERIVATIVES USEFUL AS MONOACYLGLYCEROL LIPASE INHIBITORS

(71) Applicant: Hoffmann-La Roche Inc., Little Falls, NJ (US)

(72) Inventors: Charles BELL, Cheshire (GB); Joerg BENZ, Rheinfelden (DE); Uwe GRETHER, Efringen-Kirchen (DE); Benoit HORNSPERGER, Altkirch (FR); Buelent KOCER, Maulburg (DE); Bernd KUHN, Reinach (CH); Hans RICHTER, Grenzach-Wyhlen (DE); Satoshi TSUCHIYA, Tokyo (JP)

(73) Assignee: Hoffmann-La Roche Inc., Little Falls, NJ (US)

(21) Appl. No.: 18/057,861

(22) Filed: Nov. 22, 2022

Related U.S. Application Data
(62) Division of application No. 16/899,928, filed on Jun. 12, 2020, now abandoned.
Application No. 16/899,928 is a continuation of application No. PCT/EP2018/084653, filed on Dec. 13, 2018.

(30) Foreign Application Priority Data
Dec. 15, 2017 (EP) 17207830.5

(51) Int. Cl. C07D 413/06; C07D 413/14; C07D 498/04

(52) U.S. Cl. CPC C07D 413/06; C07D 413/14; C07D 498/04

(57) ABSTRACT

The invention provides new heterocyclic compounds having the general formula (I)

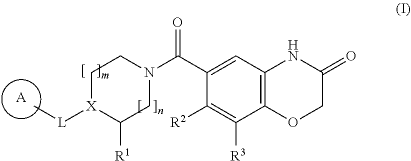

wherein A, L, X, m, n, R¹, R² and R³ are as described herein, pharmaceutically acceptable salts thereof, compositions including the compounds, processes of manufacturing the compounds and methods of using the compounds.

### CROSS REFERENCE TO RELATED APPLICATIONS

This application is a divisional of U.S. application Ser. No. 16/899,928, filed Jun. 12, 2020, which is a continuation of International Application No. PCT/EP2018/084653, filed Dec. 13, 2018, which claims priority to EP Application No. 17207830.5, filed Dec. 15, 2017, the disclosures of each of which are incorporated herein by reference in their entirety.

### FIELD OF THE INVENTION

The present invention relates to organic compounds useful for therapy or prophylaxis in a mammal, and in particular to monoacylglycerol lipase (MAGL) inhibitors for the treatment or prophylaxis of neuroinflammation, neurodegenerative diseases, pain, cancer, mental disorders, multiple sclerosis, Alzheimer's disease, Parkinson's disease, amyotrophic lateral sclerosis, traumatic brain injury, neurotoxicity, stroke, epilepsy, anxiety, migraine or depression, or any possible combination thereof, in a mammal.

### BACKGROUND OF THE INVENTION

Endocannabinoids (ECs) are signaling lipids that exert their biological actions by interacting with cannabinoid receptors (CBRs), CB1 and CB2. They modulate multiple physiological processes including neuroinflammation, neurodegeneration and tissue regeneration (Iannotti, F. A., et al., Progress in lipid research 2016, 62, 107-28.). In the brain, the main endocannabinoid, 2-arachidonoylglycerol (2-AG), is produced by diacyglycerol lipases (DAGL) and hydrolyzed by the monoacylglycerol lipase, MAGL. MAGL hydrolyses 85% of 2-AG; the remaining 15% being hydrolysed by ABHD6 and ABDH12 (Nomura, D. K., et al., Science 2011, 334, 809.). MAGL is expressed throughout the brain and in most brain cell types, including neurons, astrocytes, oligodendrocytes and microglia cells (Chanda, P. K., et al., Molecular pharmacology 2010, 78, 996; Viader, A., et al., Cell reports 2015, 12, 798.). 2-AG hydrolysis results in the formation of arachidonic acid (AA), the precursor of prostaglandins (PGs) and leukotrienes (LTs). Oxidative metabolism of AA is increased in inflamed tissues. There are two principal enzyme pathways of arachidonic acid oxygenation involved in inflammatory processes, the cyclooxygenase which produces PGs and the 5-lipoxygenase which produces LTs. Of the various cyclooxygenase products formed during inflammation, PGE2 is one of the most important. These products have been detected at sites of inflammation, e.g. in the cerebrospinal fluid of patients suffering from neurodegenerative disorders and are believed to contribute to inflammatory response and disease progression. Mice lacking MAGL (Mgll−/−) exhibit dramatically reduced 2-AG hydrolase activity and elevated 2-AG levels in the nervous system while other arachidonoyl-containing phospho- and neutral lipid species including anandamide (AEA), as well as other free fatty acids, are unaltered. Conversely, levels of AA and AA-derived prostaglandins and other eicosanoids, including prostaglandin E2 (PGE2), D2 (PGD2), F2 (PGF2), and thromboxane B2 (TXB2), are strongly decreased. Phospholipase A₂ (PLA₂) enzymes have been viewed as the principal source of AA, but cPLA₂-deficient mice have unaltered AA levels in their brain, reinforcing the key role of MAGL in the brain for AA production and regulation of the brain inflammatory process.

Neuroinflammation is a common pathological change characteristic of diseases of the brain including, but not restricted to, neurodegenerative diseases (e.g. multiple sclerosis, Alzheimer's disease, Parkinson disease, amyotrophic lateral sclerosis, traumatic brain injury, neurotoxicity, stroke, epilepsy and mental disorders such as anxiety and migraine). In the brain, production of eicosanoids and prostaglandins controls the neuroinflammation process. The pro-inflammatory agent lipopolysaccharide (LPS) produces a robust, time-dependent increase in brain eicosanoids that is markedly blunted in Mgll−/− mice. LPS treatment also induces a widespread elevation in pro-inflammatory cytokines including interleukin-1-a (IL-1-a), IL-1b, IL-6, and tumor necrosis factor-a (TNF-α) that is prevented in Mgll−/− mice.

Neuroinflammation is characterized by the activation of the innate immune cells of the central nervous system, the microglia and the astrocytes. It has been reported that anti-inflammatory drugs can suppress in preclinical models the activation of glia cells and the progression of disease including Alzheimer's disease and multiple sclerosis (Lleo A., Cell Mol Life Sci. 2007, 64, 1403.). Importantly, genetic and/or pharmacological disruption of MAGL activity also blocks LPS-induced activation of microglial cells in the brain (Nomura, D. K., et al., Science 2011, 334, 809.).

In addition, genetic and/or pharmacological disruption of MAGL activity was shown to be protective in several animal models of neurodegeneration including, but not restricted to, Alzheimer's disease, Parkinson's disease and multiple sclerosis. For example, an irreversible MAGL inhibitor has been widely used in preclinical models of neuroinflammation and neurodegeneration (Long, J. Z., et al., Nature chemical biology 2009, 5, 37.). Systemic injection of such inhibitor recapitulates the Mgll−/− mice phenotype in the brain, including an increase in 2-AG levels, a reduction in AA levels and related eicosanoids production, as well as the prevention of cytokines production and microglia activation following LPS-induced neuroinflammation (Nomura, D. K., et al., Science 2011, 334, 809.), altogether confirming that MAGL is a druggable target.

Consecutive to the genetic and/or pharmacological disruption of MAGL activity, the endogenous levels of the MAGL natural substrate in the brain, 2-AG, are increased. 2-AG has been reported to show beneficial effects on pain with, for example, anti-nociceptive effects in mice (Ignatowska-Jankowska B. et al., J Pharmacol. Exp. Ther. 2015, 353, 424.) and on mental disorders, such as depression in chronic stress models (Zhong P. et al., Neuropsychopharmacology 2014, 39, 1763.).

Furthermore, oligodendrocytes (OLs), the myelinating cells of the central nervous system, and their precursors (OPCs) express the cannabinoid receptor 2 (CB2) on their membrane. 2-AG is the endogenous ligand of CB1 and CB2 receptors. It has been reported that both cannabinoids and pharmacological inhibition of MAGL attenuate OLs's and OPCs's vulnerability to excitotoxic insults and therefore may be neuroprotective (Bernal-Chico, A., et al., Glia 2015, 63, 163.). Additionally, pharmacological inhibition of MAGL increases the number of myelinating OLs in the brain of mice, suggesting that MAGL inhibition may promote differentiation of OPCs in myelinating OLs in vivo (Alpar, A., et al., Nature communications 2014, 5, 4421.). Inhibition of MAGL was also shown to promote remyelination and functional recovery in a mouse model of progressive multiple sclerosis (Feliu A. et al., Journal of Neuroscience 2017, 37 (35), 8385.).

Finally, in recent years, metabolism is talked highly important in cancer research, especially the lipid metabolism. Researchers believe that the de novo fatty acid synthesis plays an important role in tumor development. Many studies illustrated that endocannabinoids have anti-tumorigenic actions, including anti-proliferation, apoptosis induction and anti-metastatic effects. MAGL as an important decomposing enzyme for both lipid metabolism and the endocannabinoids system, additionally as a part of a gene expression signature, contributes to different aspects of tumourigenesis (Qin, H., et al., Cell Biochem. Biophys. 2014, 70, 33; Nomura D K et al., Cell 2009, 140(1), 49-61; Nomura D K et al., Chem. Biol. 2011, 18(7), 846-856).

In conclusion, suppressing the action and/or the activation of MAGL is a promising new therapeutic strategy for the treatment or prevention of neuroinflammation, neurodegenerative diseases, pain, cancer and mental disorders. Furthermore, suppressing the action and/or the activation of MAGL is a promising new therapeutic strategy for providing neuroprotection and myelin regeneration. Accordingly, there is a high unmet medical need for new MAGL inhibitors.

### SUMMARY OF THE INVENTION

In a first aspect, the present invention provides compounds of Formula (I)

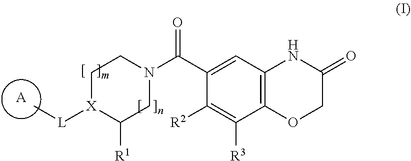

wherein:
A is selected from the group consisting of aryl, heteroaryl, cycloalkyl and heterocyclyl, wherein each of said aryl, heteroaryl, cycloalkyl and heterocyclyl is independently substituted with R⁴ and R⁵;
L is selected from the group consisting of heterocyclyl, —O—, —C(O)—, —S(O)₂—, —CHR⁶—, —CH₂CH₂—, —(CH₂)ₚ—C(O)—NR⁷— and —(CH₂)_{q}—NR⁸—C(O)—;

### X is N or C—R⁹,

each of m, n, p and q is independently an integer selected from the group consisting of 0 and 1; and
each of R¹, R², R³, R⁴, R⁵, R⁶, R⁷, R⁸, and R⁹ is independently selected from the group consisting of hydrogen, hydroxy, halogen, cyano, alkyl, haloalkyl, cycloalkyl, alkoxy, haloalkoxy, aryl and heteroaryl;
or pharmaceutically acceptable salts thereof.

In a further aspect, the present invention provides a process of manufacturing the compounds of formula (I) as described herein, comprising the steps of:

a) reacting an amine 1, wherein A, L, X, R¹, m and n are as described herein,

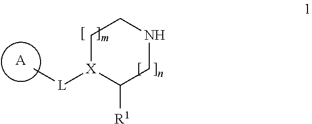

with an acid 2, wherein R² and R³ are as described herein

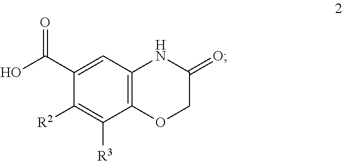

or

b) reacting an amine 1, wherein A, L, X, R¹, m and n are as described herein,

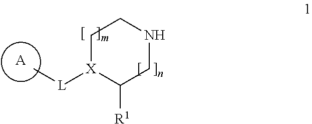

with an acid chloride 2a, wherein R² and R³ are as described herein

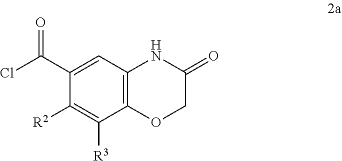

to form said compound of formula (I).

In a further aspect, there is provided a process of manufacturing compounds of formula (I), wherein said compounds of formula (I) are compounds of formula (Ia) as described herein, comprising reacting an amine 3, wherein R¹, R², R³, m and n are as defined herein,

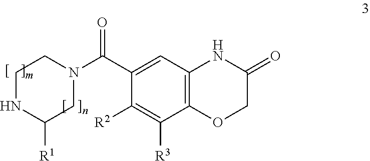

with a compound of formula 4, wherein A, L¹ and R⁶ are as defined herein,

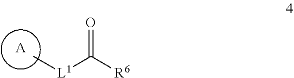

in the presence of a reducing agent, to form said compound of formula (Ia).

In a further aspect, the present invention provides a compound of formula (I) as described herein, when manufactured according to the processes described herein.

In a further aspect, the present invention provides a compound of formula (I) as described herein, or a pharmaceutically acceptable salt thereof, for use as therapeutically active substance.

In a further aspect, the present invention provides a pharmaceutical composition comprising a compound of formula (I) as described herein, or a pharmaceutically acceptable salt thereof, and a therapeutically inert carrier.

In a further aspect, the present invention provides the use of a compound of formula (I) as described herein, or a pharmaceutically acceptable salt thereof, for inhibiting monoacylglycerol lipase (MAGL) in a mammal.

In a further aspect, the present invention provides the use of a compound of formula (I) as described herein, or a pharmaceutically acceptable salt thereof, for the treatment or prophylaxis of neuroinflammation, neurodegenerative diseases, pain, cancer or mental disorders, or any possible combination thereof, in a mammal.

In a further aspect, the present invention provides the use of a compound of formula (I) as described herein, or a pharmaceutically acceptable salt thereof, for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, cancer or pain, or any possible combination thereof, in a mammal.

In a further aspect, the present invention provides a compound of formula (I) as described herein, or a pharmaceutically acceptable salt thereof, for use in a method of inhibiting monoacylglycerol lipase in a mammal.

In a further aspect, the present invention provides a compound of formula (I) as described herein, or a pharmaceutically acceptable salt thereof, for use in the treatment or prophylaxis of neuroinflammation, neurodegenerative diseases, pain, cancer or mental disorders, or any possible combination thereof, in a mammal.

In a further aspect, the present invention provides a compound of formula (I) as described herein, or a pharmaceutically acceptable salt thereof, for use in the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, cancer or pain, or any possible combination thereof, in a mammal.

In a further aspect, the present invention provides the use of a compound of formula (I) as described herein, or a pharmaceutically acceptable salt thereof, for the preparation of a medicament for inhibiting monoacylglycerol lipase in a mammal.

In a further aspect, the present invention provides the use of a compound of formula (I) as described herein, or a pharmaceutically acceptable salt thereof, for the preparation of a medicament for the treatment or prophylaxis of neuroinflammation, neurodegenerative diseases, pain, cancer or mental disorders, or any possible combination thereof, in a mammal.

In a further aspect, the present invention provides the use of a compound of formula (I) as described herein, or a pharmaceutically acceptable salt thereof, for the preparation of a medicament for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, cancer or pain, or any possible combination thereof, in a mammal.

In a further aspect, the present invention provides a method for inhibiting monoacylglycerol lipase in a mammal, which method comprises administering an effective amount of a compound of formula (I) as described, or a pharmaceutically acceptable salt thereof, herein to the mammal.

In a further aspect, the present invention provides a method for the treatment or prophylaxis of neuroinflammation, neurodegenerative diseases, pain, cancer or mental disorders, or any possible combination thereof, in a mammal, which method comprises administering an effective amount of a compound of formula (I) as described herein, or a pharmaceutically acceptable salt thereof, to the mammal.

In a further aspect, the present invention provides a method for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, cancer or pain, or any possible combination thereof, in a mammal, which method comprises administering an effective amount of a compound of formula (I) as described herein, or a pharmaceutically acceptable salt thereof, to the mammal.

### DETAILED DESCRIPTION OF THE INVENTION

### Definitions

Features, integers, characteristics, compounds, chemical moieties or groups described in conjunction with a particular aspect, embodiment or example of the invention are to be understood to be applicable to any other aspect, embodiment or example described herein, unless incompatible therewith. All of the features disclosed in this specification (including any accompanying claims, abstract and drawings), and/or all of the steps of any method or process so disclosed, may be combined in any combination, except combinations where at least some of such features and/or steps are mutually exclusive. The invention is not restricted to the details of any foregoing embodiments. The invention extends to any novel one, or any novel combination, of the features disclosed in this specification (including any accompanying claims, abstract and drawings), or to any novel one, or any novel combination, of the steps of any method or process so disclosed.

The term “alkyl” refers to a mono- or multivalent, e.g., a mono- or bivalent, linear or branched saturated hydrocarbon group of 1 to 12 carbon atoms. In some preferred embodiments, the alkyl group contains 1 to 6 carbon atoms, e.g., 1, 2, 3, 4, 5, or 6 carbon atoms. In other embodiments, the alkyl group contains 1 to 3 carbon atoms, e.g., 1, 2 or 3 carbon atoms. Examples of such groups include, but are not limited to, methyl, ethyl, propyl, 2-propyl (isopropyl), n-butyl, iso-butyl, sec-butyl, tert-butyl, and 2,2-dimethylpropyl. In a particularly preferred embodiment, alkyl is methyl.

The term “alkoxy” refers to an alkyl group, as previously defined, attached to the parent molecular moiety via an oxygen atom. Unless otherwise specified, the alkoxy group contains 1 to 12 carbon atoms. In some embodiments, the alkoxy group contains 1 to 6 carbon atoms. In other embodiments, the alkoxy group contains 1 to 4 carbon atoms. In still other embodiments, the alkoxy group contains 1 to 3 carbon atoms. Some non-limiting examples of alkoxy groups include methoxy, ethoxy, n-propoxy, isopropoxy, n-butoxy, isobutoxy and tert-butoxy. In a preferred embodiment, alkoxy is methoxy, ethoxy and isopropoxy. In a particularly preferred embodiment, alkoxy is methoxy.

The term “halogen” or “halo” refers to fluoro (F), chloro (Cl), bromo (Br), or iodo (I). In a preferred embodiment, the term “halogen” or “halo” refers to fluoro (F), chloro (Cl) or bromo (Br). In a particularly preferred embodiment, “halogen” or “halo” is fluoro (F).

The term “haloalkyl” or “haloalkoxy”, respectively, refers to an alkyl or alkoxy group, as the case may be, substituted with one or more halogen atoms, wherein each of the alkyl or alkoxy is defined as described herein. In a preferred embodiment, the haloalkyl or haloalkoxy group, respectively, contains 1, 2 or 3 halogen atoms, most preferably 1, 2 or 3 F atoms. Examples of such groups include, but are not limited to, fluoromethyl, difluoromethyl, trifluoromethyl (CF₃), 2,2,2-trifluoroethyl, trifluoromethoxy, 2,2,2-trifluoroethoxy, 2,2,3,3-tetrafluoropropoxy, and the like. A particularly preferred haloalkyl group is trifluoromethyl (CF₃).

The terms “cycloalkyl” and “carbocycle” are used herein synonymously and refer to a saturated or partly unsaturated monocyclic or bicyclic hydrocarbon group of 3 to 10 ring carbon atoms. In some embodiments, the cycloalkyl group is a saturated monocyclic hydrocarbon group of 3 to 8 ring carbon atoms. “Bicyclic cycloalkyl” refers to cycloalkyl moieties consisting of two saturated carbocycles having two carbon atoms in common, i.e., the bridge separating the two rings is either a single bond or a chain of one or two ring atoms, and to spirocyclic moieties, i.e., the two rings are connected via one common ring atom. In a preferred embodiment, the cycloalkyl group is a saturated monocyclic hydrocarbon group of 3 to 6 ring carbon atoms, e.g., of 3, 4, 5 or 6 carbon atoms. In a preferred embodiment, the term “cycloalkyl” refers to cyclopropyl.

The terms “heterocyclyl” and “heterocycle” are used herein synonymously and refer to a saturated or partly unsaturated mono- or bicyclic ring system of 3 to 10 ring atoms, wherein 1, 2, or 3 of said ring atoms are heteroatoms selected from the group consisting of N, O and S, the remaining ring atoms being carbon. Preferably, 1, 2, or 3 of said ring atoms are N, the remaining ring atoms being carbon “Bicyclic heterocyclyl” refers to heterocyclic moieties consisting of two cycles having two ring atoms in common, i.e., the bridge separating the two rings is either a single bond or a chain of one or two ring atoms, and to spirocyclic moieties, i.e., the two rings are connected via one common ring atom. Examples for monocyclic heterocyclyl groups include, but are not limited to, 1-piperidyl, 2-piperidyl, 3-piperidyl, 4-piperidyl, oxazolidine-2-one, oxazolidine-4-one and oxazolidine-5-one.

The term “aryl” refers to a monocyclic, bicyclic, or tricyclic carbocyclic ring system having a total of 6 to 14 ring members, preferably, 6 to 12 ring members, and more preferably 6 to 10 ring members, and wherein at least one ring in the system is aromatic. A particular, yet non-limiting example of aryl is phenyl.

The term “heteroaryl” refers to a mono- or multivalent, monocyclic or bicyclic ring system having a total of 5 to 12 ring members, preferably, 5 to 10 ring members, and more preferably 5 to 6 ring members, wherein at least one ring in the system is aromatic, and at least one ring in the system contains one or more heteroatoms. In one embodiment, a 5-10 membered heteroaryl comprises 1, 2, 3 or 4 heteroatoms independently selected from the group consisting of O, S and N. Some non-limiting examples of heteroaryl rings include 2-pyridyl, 3-pyridyl and 4-pyridyl.

The term “hydroxy” refers to an —OH group.

The term “cyano” refers to a —CN group.

The term “pharmaceutically acceptable salt” refers to those salts which retain the biological effectiveness and properties of the free bases or free acids, which are not biologically or otherwise undesirable. The salts are formed with inorganic acids such as hydrochloric acid, hydrobromic acid, sulfuric acid, nitric acid, phosphoric acid and the like, in particular hydrochloric acid, and organic acids such as acetic acid, propionic acid, glycolic acid, pyruvic acid, oxalic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, N-acetylcystein and the like. In addition these salts may be prepared by addition of an inorganic base or an organic base to the free acid. Salts derived from an inorganic base include, but are not limited to, the sodium, potassium, lithium, ammonium, calcium, magnesium salts and the like. Salts derived from organic bases include, but are not limited to salts of primary, secondary, and tertiary amines, substituted amines including naturally occurring substituted amines, cyclic amines and basic ion exchange resins, such as isopropylamine, trimethylamine, diethylamine, triethylamine, tripropylamine, ethanolamine, lysine, arginine, N-ethylpiperidine, piperidine, polyimine resins and the like. Particular pharmaceutically acceptable salts of compounds of formula (I) are hydrochloride salts.

The term “protecting group” (PG) denotes the group which selectively blocks a reactive site in a multifunctional compound such that a chemical reaction can be carried out selectively at another unprotected reactive site in the meaning conventionally associated with it in synthetic chemistry. Protecting groups can be removed at the appropriate point. Exemplary protecting groups are amino-protecting groups, carboxy-protecting groups or hydroxy-protecting groups. Particular protecting groups are the tert-butoxycarbonyl (Boc), benzyloxycarbonyl (Cbz), fluorenylmethoxycarbonyl (Fmoc) and benzyl (Bn). Further particular protecting groups are the tert-butoxycarbonyl (Boc) and the fluorenylmethoxycarbonyl (Fmoc). More particular protecting group is the tert-butoxycarbonyl (Boc). Exemplary protecting groups and their application in organic synthesis are described, for example, in “Protective Groups in Organic Chemistry” by T. W. Greene and P. G. M. Wutts, 5th Ed., 2014, John Wiley & Sons, N.Y.

The compounds of formula (I) can contain several asymmetric centers and can be present in the form of optically pure enantiomers, mixtures of enantiomers such as, for example, racemates, optically pure diastereioisomers, mixtures of diastereoisomers, diastereoisomeric racemates or mixtures of diastereoisomeric racemates.

According to the Cahn-Ingold-Prelog Convention, the asymmetric carbon atom can be of the “R” or “S” configuration.

The abbreviation “MAGL” refers to the enzyme monoacylglycerol lipase. The terms “MAGL” and “monoacylglycerol lipase” are used herein interchangeably.

The term “treatment” as used herein includes: (1) inhibiting the state, disorder or condition (e.g. arresting, reducing or delaying the development of the disease, or a relapse thereof in case of maintenance treatment, of at least one clinical or subclinical symptom thereof); and/or (2) relieving the condition (i.e., causing regression of the state, disorder or condition or at least one of its clinical or subclinical symptoms). The benefit to a patient to be treated is either statistically significant or at least perceptible to the patient or to the physician. However, it will be appreciated that when a medicament is administered to a patient to treat a disease, the outcome may not always be effective treatment.

The term “prophylaxis” as used herein includes: preventing or delaying the appearance of clinical symptoms of the state, disorder or condition developing in a mammal and especially a human that may be afflicted with or predisposed to the state, disorder or condition but does not yet experience or display clinical or subclinical symptoms of the state, disorder or condition.

The term “neuroinflammation” as used herein relates to acute and chronic inflammation of the nervous tissue, which is the main tissue component of the two parts of the nervous system; the brain and spinal cord of the central nervous system (CNS), and the branching peripheral nerves of the peripheral nervous system (PNS). Chronic neuroinflammation is associated with neurodegenerative diseases such as Alzheimer's disease, Parkinson's disease and multiple sclerosis. Acute neuroinflammation usually follows injury to the central nervous system immediately, e.g., as a result of traumatic brain injury (TBI).

The term “traumatic brain injury” (“TBI”, also known as “intracranial injury”), relates to damage to the brain resulting from external mechanical force, such as rapid acceleration or deceleration, impact, blast waves, or penetration by a projectile.

The term “neurodegenerative diseases” relates to diseases that are related to the progressive loss of structure or function of neurons, including death of neurons. Examples of neurodegenerative diseases include, but are not limited to, multiple sclerosis, Alzheimer's disease, Parkinson's disease and amyotrophic lateral sclerosis.

The term “mental disorders” (also called mental illnesses or psychiatric disorders) relates to behavioral or mental patterns that may cause suffering or a poor ability to function in life. Such features may be persistent, relapsing and remitting, or occur as a single episode. Examples of mental disorders include, but are not limited to, anxiety and depression.

The term “pain” relates to an unpleasant sensory and emotional experience associated with actual or potential tissue damage. Examples of pain include, but are not limited to, nociceptive pain, chronic pain (including idiopathic pain), neuropathic pain, phantom pain and phsychogenic pain. A particular example of pain is neuropathic pain, which is caused by damage or disease affecting any part of the nervous system involved in bodily feelings (i.e., the somatosensory system). In one embodiment, “pain” is neuropathic pain resulting from amputation or thoracotomy.

The term “neurotoxicity” relates to toxicity in the nervous system. It occurs when exposure to natural or artificial toxic substances (neurotoxins) alter the normal activity of the nervous system in such a way as to cause damage to nervous tissue. Examples of neurotoxicity include, but are not limited to, neurotoxicity resulting from exposure to substances used in chemotherapy, radiation treatment, drug therapies, drug abuse, and organ transplants, as well as exposure to heavy metals, certain foods and food additives, pesticides, industrial and/or cleaning solvents, cosmetics, and some naturally occurring substances.

The term “mammal” as used herein includes both humans and non-humans and includes but is not limited to humans, non-human primates, canines, felines, murines, bovines, equines, and porcines. In a particularly preferred embodiment, the term “mammal” refers to humans.

### Compounds of the Invention

In a first aspect, the present invention provides compounds of Formula (I)

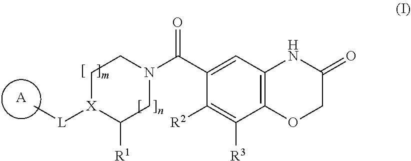

wherein:
A is selected from the group consisting of aryl, heteroaryl, cycloalkyl and heterocyclyl, wherein each of said aryl, heteroaryl, cycloalkyl and heterocyclyl is independently substituted with R⁴ and R⁵;
L is selected from the group consisting of heterocyclyl, —O—, —C(O)—, —S(O)₂—, —CHR⁶—, —CH₂CH₂—, —(CH₂)ₚ—C(O)—NR⁷— and —(CH₂)_{q}—NR⁸—C(O)—;

### X is N or C—R⁹,

each of m, n, p and q is independently an integer selected from the group consisting of 0 and 1; and
each of R¹, R², R³, R⁴, R⁵, R⁶, R⁷, R⁸, and R⁹ is independently selected from the group consisting of hydrogen, hydroxy, halogen, cyano, alkyl, haloalkyl, cycloalkyl, alkoxy, haloalkoxy, aryl and heteroaryl;
or pharmaceutically acceptable salts thereof.

In one embodiment, there is provided a compound of formula (I) as described herein, or a pharmaceutically acceptable salt thereof, wherein A is selected from the group consisting of aryl, heteroaryl and heterocyclyl, wherein each of said aryl, heteroaryl and heterocyclyl is independently substituted with R⁴ and R⁵;

R⁴ is selected from the group consisting of hydrogen, halogen, haloalkyl, alkoxy, cyano and aryl; and
R⁵ is hydrogen or halogen.

In one embodiment, there is provided a compound of formula (I) as described herein, or a pharmaceutically acceptable salt thereof, wherein A is aryl substituted with R⁴ and R⁵;

R⁴ is selected from the group consisting of hydrogen, halogen, haloalkyl, alkoxy, cyano and aryl; and
R⁵ is hydrogen or halogen.

In one embodiment, there is provided a compound of formula (I) as described herein, or a pharmaceutically acceptable salt thereof, wherein A is heteroaryl substituted with R⁴ and R⁵; and

R⁴ and R⁵ are both hydrogen.

In one embodiment, there is provided a compound of formula (I) as described herein, or a pharmaceutically acceptable salt thereof, wherein A is heterocyclyl substituted with R⁴ and R⁵; and

R⁴ and R⁵ are both hydrogen.

In a preferred embodiment, there is provided a compound of formula (I) as described herein, or a pharmaceutically acceptable salt thereof, wherein A is aryl substituted with R⁴ and R⁵;

R⁴ is selected from the group consisting of hydrogen, halogen, haloalkyl and alkoxy; and
R⁵ is hydrogen or halogen.

In a particularly preferred embodiment, there is provided a compound of formula (I) as described herein, or a pharmaceutically acceptable salt thereof, wherein A is phenyl substituted with R⁴ and R⁵;

R⁴ is selected from the group consisting of hydrogen, fluorine, chlorine, trifluoromethyl and methoxy; and
R⁵ is hydrogen or fluorine.

In a further particularly preferred embodiment, there is provided a compound of formula (I) as described herein, or a pharmaceutically acceptable salt thereof, wherein A is selected from the group consisting of phenyl, 4-fluorophenyl, 3-fluorophenyl, 4-chlorophenyl, 3-(trifluoromethyl)phenyl, 4-(trifluoromethyl)phenyl, 3-methoxyphenyl, 3,5-difluorophenyl and 2,3-difluorophenyl.

In one embodiment, there is provided a compound of formula (I) as described herein, or a pharmaceutically acceptable salt thereof, wherein L is selected from the group consisting of heterocyclyl, —O—, —C(O)—, —S(O)₂—, —CHR⁶—, —CH₂CH₂— and —(CH₂)ₚ—C(O)—NR⁷—;

p is 0 or 1;
R⁶ is hydrogen or halogen; and
R⁷ is hydrogen, alkyl or cycloalkyl.

In one embodiment, there is provided a compound of formula (I) as described herein, or a pharmaceutically acceptable salt thereof, wherein L is selected from the group consisting of heterocyclyl, —O—, —C(O)—, —S(O)₂—, —CHR⁶—, —CH₂CH₂— and —(CH₂)ₚ—C(O)—NR⁷—; and

X is N or C—R⁹, provided that:
when L is —(CH₂)ₚ—C(O)—NR⁷—, X is C—R⁹.

In one embodiment, there is provided a compound of formula (I) as described herein, or a pharmaceutically acceptable salt thereof, wherein L is selected from the group consisting of heterocyclyl, —O—, —C(O)—, —S(O)₂—, —CH₂—, —CH₂CH₂— and —(CH₂)ₚ—C(O)—NR⁷—;

p is 0 or 1; and
R⁷ is alkyl or cycloalkyl.

In a preferred embodiment, there is provided a compound of formula (I) as described herein, or a pharmaceutically acceptable salt thereof, wherein L is —O— or —CH₂—.

In a particularly preferred embodiment, there is provided a compound of formula (I) as described herein, or a pharmaceutically acceptable salt thereof, wherein L is —O—.

In a further particularly preferred embodiment, there is provided a compound of formula (I) as described herein, or a pharmaceutically acceptable salt thereof, wherein L is —CH₂.

In a preferred embodiment, there is provided a compound of formula (I) as described herein, or a pharmaceutically acceptable salt thereof, wherein X is N or C—R⁹; and R⁹ is selected from the group consisting of hydrogen, hydroxy and halogen.

In a particularly preferred embodiment, there is provided a compound of formula (I) as described herein, or a pharmaceutically acceptable salt thereof, wherein X is C—H.

In one embodiment, there is provided a compound of formula (I) as described herein, or a pharmaceutically acceptable salt thereof, wherein m and n are both 1.

In a preferred embodiment, there is provided a compound of formula (I) as described herein, or a pharmaceutically acceptable salt thereof, wherein R¹ is selected from the group consisting of hydrogen, alkyl, haloalkyl and heteroaryl.

In a particularly preferred embodiment, there is provided a compound of formula (I) as described herein, or a pharmaceutically acceptable salt thereof, wherein R¹ is hydrogen.

In a further particularly preferred embodiment, there is provided a compound of formula (I) as described herein, or a pharmaceutically acceptable salt thereof, wherein R² is hydrogen.

In yet a further particularly preferred embodiment, there is provided a compound of formula (I) as described herein, or a pharmaceutically acceptable salt thereof, wherein R³ is hydrogen.

In one embodiment, there is provided a compound of formula (I) as described herein, or a pharmaceutically acceptable salt thereof, wherein:

A is selected from the group consisting of aryl, heteroaryl and heterocyclyl, wherein each of said aryl, heteroaryl and heterocyclyl is independently substituted with R⁴ and R⁵;
L is selected from the group consisting of heterocyclyl, —O—, —C(O)—, —S(O)₂—, —CH₂—, —CH₂CH₂—, and —(CH₂)ₚ—C(O)—NR⁷—;

### X is N or C—R⁹,

each of m, n and p is independently an integer selected from the group consisting of 0 and 1;
R¹ is selected from the group consisting of hydrogen, alkyl, haloalkyl and heteroaryl;
R² and R³ are both hydrogen;
R⁴ is selected from the group consisting of hydrogen, halogen, haloalkyl, alkoxy, cyano and aryl;
R⁵ is hydrogen or halogen;
R⁷ is alkyl or cycloalkyl; and
R⁹ is selected from the group consisting of hydrogen, hydroxy and halogen.

In a preferred embodiment, there is provided a compound of formula (I) as described herein, or a pharmaceutically acceptable salt thereof, wherein:

A is aryl substituted with R⁴ and R⁵;

### L is —O— or —CH₂—;

### X is C—H,

m and n are both 1;
each of R¹, R² and R³ is hydrogen;
R⁴ is selected from the group consisting of hydrogen, halogen, haloalkyl and alkoxy; and
R⁵ is hydrogen or halogen.

In a particularly preferred embodiment, there is provided a compound of formula (I) as described herein, or a pharmaceutically acceptable salt thereof, wherein:

A is phenyl substituted with R⁴ and R⁵;

### L is —O— or —CH₂—;

### X is C—H,

m and n are both 1;
each of R¹, R² and R³ is hydrogen;
R⁴ is selected from the group consisting of hydrogen, fluorine, chlorine, trifluoromethyl and methoxy; and
R⁵ is hydrogen or fluorine.

In a further particularly preferred embodiment, there is provided a compound of formula (I) as described herein, or a pharmaceutically acceptable salt thereof, wherein:

A is selected from the group consisting of phenyl, 4-fluorophenyl, 3-fluorophenyl, 4-chlorophenyl, 3-(trifluoromethyl)phenyl, 4-(trifluoromethyl)phenyl, 3-methoxyphenyl, 3,5-difluorophenyl and 2,3-difluorophenyl;

### L is —O— or —CH₂—;

### X is C—H,

m and n are both 1; and
each of R¹, R² and R³ is hydrogen.

In one embodiment, the compound of formula (I) is a compound of formula (Ia), or a pharmaceutically acceptable salt thereof,

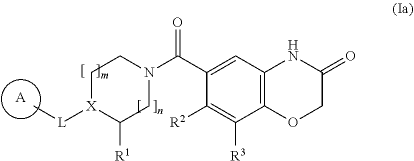

wherein A, m, n, R¹, R² and R³ are as defined herein in relation to compounds of formula (I) and L is selected from the group consisting of heterocyclyl, —CHR⁶— and —CH₂CH₂—, preferably from —CHR⁶— and —CH₂CH₂—, wherein R⁶ is selected from the group consisting of hydrogen, alkyl, haloalkyl, cycloalkyl, aryl and heteroaryl, preferably from hydrogen and alkyl, in particular wherein R⁶ is hydrogen.

In a preferred embodiment, the compound of formula (I) is a compound of formula (Ia) as described herein, wherein R¹ is selected from the group consisting of alkyl, haloalkyl and heteroaryl and L is —CH₂— or —CH₂CH₂—.

In one embodiment, there is provided a compound of formula (I) as described herein, or a pharmaceutically acceptable salt thereof, wherein said compound of formula (I) is selected from the group consisting of.
6-(4-benzylpiperidine-1-carbonyl)-4H-1,4-benzoxazin-3-one;
6-(4-benzylpiperazine-1-carbonyl)-4H-1,4-benzoxazin-3-one;
6-[4-[(4-fluorophenyl)methyl]piperidine-1-carbonyl]-4H-1,4-benzoxazin-3-one;
6-[4-[[4-(trifluoromethyl)phenyl]methyl]piperidine-1-carbonyl]-4H-1,4-benzoxazin-3-one;
6-[4-[(4-methoxyphenyl)methyl]piperidine-1-carbonyl]-4H-1,4-benzoxazin-3-one;
6-[4-[(3-methoxyphenyl)methyl]piperidine-1-carbonyl]-4H-1,4-benzoxazin-3-one;
6-(4-benzyl-4-hydroxypiperidine-1-carbonyl)-4H-1,4-benzoxazin-3-one;
6-[4-[(3-fluorophenyl)methyl]piperidine-1-carbonyl]-4H-1,4-benzoxazin-3-one;
6-[4-[(3,5-difluorophenyl)methyl]piperidine-1-carbonyl]-4H-1,4-benzoxazin-3-one;
6-[4-(benzenesulfonyl)piperidine-1-carbonyl]-4H-1,4-benzoxazin-3-one;
6-[4-[(5S)-2-oxo-5-phenyl-1,3-oxazolidin-3-yl]piperidine-1-carbonyl]-4H-1,4-benzoxazin-3-one;
6-[4-[(4-chlorophenyl)methyl]piperidine-1-carbonyl]-4H-1,4-benzoxazin-3-one;
6-(4-benzoylpiperidine-1-carbonyl)-4H-1,4-benzoxazin-3-one;
6-(4-phenoxypiperidine-1-carbonyl)-4H-1,4-benzoxazin-3-one;
6-[4-[4-(trifluoromethyl)phenoxy]piperidine-1-carbonyl]-4H-1,4-benzoxazin-3-one;
6-[4-[(2,3-difluorophenyl)methyl]piperidine-1-carbonyl]-4H-1,4-benzoxazin-3-one;
6-[4-[[3-(trifluoromethyl)phenyl]methyl]piperidine-1-carbonyl]-4H-1,4-benzoxazin-3-one;
6-(3-phenoxypyrrolidine-1-carbonyl)-4H-1,4-benzoxazin-3-one;
6-[4-[(5R)-2-oxo-5-phenyl-1,3-oxazolidin-3-yl]piperidine-1-carbonyl]-4H-1,4-benzoxazin-3-one;
4-[[1-(3-oxo-4H-1,4-benzoxazine-6-carbonyl)piperidin-4-yl]methyl]benzonitrile;
6-[4-[(2-phenylphenyl)methyl]piperidine-1-carbonyl]-4H-1,4-benzoxazin-3-one;
6-[4-(pyridin-2-ylmethyl)piperidine-1-carbonyl]-4H-1,4-benzoxazin-3-one;
6-[4-[(4-chlorophenyl)methyl]-4-fluoropiperidine-1-carbonyl]-4H-1,4-benzoxazin-3-one;
6-(4-benzylpiperidine-1-carbonyl)-8-fluoro-4H-1,4-benzoxazin-3-one;
N-methyl-N-[1-(3-oxo-4H-1,4-benzoxazine-6-carbonyl)piperidin-4-yl]benzamide;
6-(4-benzylpiperidine-1-carbonyl)-4H-pyrido[3,2-b][1,4]oxazin-3-one;
6-[4-(piperidine-1-carbonyl)piperidine-1-carbonyl]-4H-1,4-benzoxazin-3-one;
6-(3-benzylpyrrolidine-1-carbonyl)-4H-1,4-benzoxazin-3-one;
6-[3-[[4-(trifluoromethyl)phenyl]methyl]azetidine-1-carbonyl]-4H-1,4-benzoxazin-3-one;
6-[3-[[3-(trifluoromethyl)phenyl]methyl]pyrrolidine-1-carbonyl]-4H-1,4-benzoxazin-3-one;
6-[3-[[4-(trifluoromethyl)phenyl]methyl]pyrrolidine-1-carbonyl]-4H-1,4-benzoxazin-3-one;
6-[4-[4-(trifluoromethyl)benzoyl]piperazine-1-carbonyl]-4H-1,4-benzoxazin-3-one;
N-cyclopropyl-N-[1-(3-oxo-4H-1,4-benzoxazine-6-carbonyl)piperidin-4-yl]-2-phenylacetamide;
6-[4-[2-(3-chlorophenyl)ethyl]-3-(1H-pyrazol-3-yl)piperazine-1-carbonyl]-4H-1,4-benzoxazin-3-one;
6-[3-(trifluoromethyl)-4-[[4-(trifluoromethyl)phenyl]methyl]piperazine-1-carbonyl]-4H-1,4-benzoxazin-3-one;
6-[3-(1H-pyrazol-3-yl)-4-[[4-(trifluoromethyl)phenyl]methyl]piperazine-1-carbonyl]-4H-1,4-benzoxazin-3-one; and
6-[3-methyl-4-[[4-(trifluoromethyl)phenyl]methyl]piperazine-1-carbonyl]-4H-1,4-benzoxazin-3-one.

In one embodiment, there is provided a compound of formula (I) as described herein, or a pharmaceutically acceptable salt thereof, wherein said compound of formula (I) is selected from the group consisting of:
6-(4-benzylpiperidine-1-carbonyl)-4H-1,4-benzoxazin-3-one;
6-[4-[(4-fluorophenyl)methyl]piperidine-1-carbonyl]-4H-1,4-benzoxazin-3-one;
6-[4-[[4-(trifluoromethyl)phenyl]methyl]piperidine-1-carbonyl]-4H-1,4-benzoxazin-3-one;
6-[4-[(3-methoxyphenyl)methyl]piperidine-1-carbonyl]-4H-1,4-benzoxazin-3-one;
6-[4-[(3-fluorophenyl)methyl]piperidine-1-carbonyl]-4H-1,4-benzoxazin-3-one;
6-[4-[(3,5-difluorophenyl)methyl]piperidine-1-carbonyl]-4H-1,4-benzoxazin-3-one;
6-[4-[(4-chlorophenyl)methyl]piperidine-1-carbonyl]-4H-1,4-benzoxazin-3-one;
6-[4-[4-(trifluoromethyl)phenoxy]piperidine-1-carbonyl]-4H-1,4-benzoxazin-3-one;
6-[4-[(2,3-difluorophenyl)methyl]piperidine-1-carbonyl]-4H-1,4-benzoxazin-3-one; and
6-[4-[[3-(trifluoromethyl)phenyl]methyl]piperidine-1-carbonyl]-4H-1,4-benzoxazin-3-one.

In a preferred embodiment, the compound of formula (I) is a compound of formula (Ia), or a pharmaceutically acceptable salt thereof, wherein said compound of formula (Ia) is selected from the group consisting of:
6-[4-[2-(3-chlorophenyl)ethyl]-3-(1H-pyrazol-3-yl)piperazine-1-carbonyl]-4H-1,4-benzoxazin-3-one;
6-[3-(trifluoromethyl)-4-[[4-(trifluoromethyl)phenyl]methyl]piperazine-1-carbonyl]-4H-1,4-benzoxazin-3-one;
6-[3-(1H-pyrazol-3-yl)-4-[[4-(trifluoromethyl)phenyl]methyl]piperazine-1-carbonyl]-4H-1,4-benzoxazin-3-one; and
6-[3-methyl-4-[[4-(trifluoromethyl)phenyl]methyl]piperazine-1-carbonyl]-4H-1,4-benzoxazin-3-one.

In a particular embodiment, the present invention provides pharmaceutically acceptable salts of the compounds according to formula (I) as described herein, especially hydrochloride salts. In a further particular embodiment, the present invention provides compounds according to formula (I) as described herein.

### Processes of Manufacturing

The preparation of compounds of formula (I) of the present invention may be carried out in sequential or convergent synthetic routes. Syntheses of the invention are shown in the following general schemes. The skills required for carrying out the reaction and purification of the resulting products are known to those persons skilled in the art. The substituents and indices used in the following description of the processes have the significance given herein, unless indicated to the contrary.

If one of the starting materials, intermediates or compounds of formula (I) contain one or more functional groups which are not stable or are reactive under the reaction conditions of one or more reaction steps, appropriate protecting groups (as described e.g., in “Protective Groups in Organic Chemistry” by T. W. Greene and P. G. M. Wutts, 5th Ed., 2014, John Wiley & Sons, N.Y.) can be introduced before the critical step applying methods well known in the art. Such protecting groups can be removed at a later stage of the synthesis using standard methods described in the literature.

If starting materials or intermediates contain stereogenic centers, compounds of formula (I) can be obtained as mixtures of diastereomers or enantiomers, which can be separated by methods well known in the art e.g., chiral HPLC, chiral SFC or chiral crystallization. Racemic compounds can e.g., be separated into their antipodes via diastereomeric salts by crystallization with optically pure acids or by separation of the antipodes by specific chromatographic methods using either a chiral adsorbent or a chiral eluent.

A person skilled in the art will acknowledge that in the synthesis of compounds of formula (I)—insofar not desired otherwise—an “orthogonal protection group strategy” will be applied, allowing the cleavage of several protective groups one at a time each without affecting other protecting groups in the molecule. The principle of orthogonal protection is well known in the art and has also been described in literature (e.g. Barany and R. B. Merrifield, J. Am. Chem. Soc. 1977, 99, 7363; H. Waldmann et al., Angew. Chem. Int. Ed. Engl. 1996, 35, 2056).

A person skilled in the art will acknowledge that the sequence of reactions may be varied depending on reactivity and nature of the intermediates.

In more detail, the compounds of formula (I) can be manufactured by the methods given below, by the methods given in the examples or by analogous methods. Appropriate reaction conditions for the individual reaction steps are known to a person skilled in the art. Also, for reaction conditions described in literature affecting the described reactions see for example: Comprehensive Organic Transformations: A Guide to Functional Group Preparations, 2nd Edition, Richard C. Larock. John Wiley & Sons, New York, N.Y. 1999). It was found convenient to carry out the reactions in the presence or absence of a solvent. There is no particular restriction on the nature of the solvent to be employed, provided that it has no adverse effect on the reaction or the reagents involved and that it can dissolve the reagents, at least to some extent. The described reactions can take place over a wide range of temperatures, and the precise reaction temperature is not critical to the invention. It is convenient to carry out the described reactions in a temperature range between −78° C. to reflux. The time required for the reaction may also vary widely, depending on many factors, notably the reaction temperature and the nature of the reagents. However, a period of from 0.5 hours to several days will usually suffice to yield the described intermediates and compounds. The reaction sequence is not limited to the one displayed in the schemes, however, depending on the starting materials and their respective reactivity, the sequence of reaction steps can be freely altered.

The following abbreviations are used in the present text:

AcOH=acetic acid, aq.=aqueous, Boc=tert-butyloxycarbonyl, CAS RN=chemical abstracts registration number, CDI=1,1′-Carbonyldiimidazole, CHCl₃=Chloroform, DCM=dichloromethane, DCE=1,2-dichloroethane, DCC=N,N′-dicyclohexylcarbodiimide, DMA=N,N-dimethylacetamide, DMAP=4-dimethylaminopyridine, DMF=N,N-dimethylformamide, EDCI=N-(3-dimethylaminopropyl)-N′-ethylcarbodiimide hydrochloride, EtOAc=ethylacetate, EtOH=ethanol, HATU=O-(7-azabenzotriazol-1-yl)-1,1,3,3-tetramethyluronium hexafluorophosphate, HCl=hydrogen chloride, HPLC=high performance liquid chromatography, HOBt=1-hydroxybenzo-triazole, Huenig's base=iPr₂NEt=N-ethyl diisopropylamine, K₂CO₃=potassium carbonate, MeOH=methanol, RT=room temperature, MeI=methyl iodide, MS=mass spectrum, NaHCO₃=sodium hydrogen carbonate, Na₂CO₃=sodium carbonate, Na₂SO₄=sodium sulfate, sat.=saturated, SFC==Supercritical Fluid Chromatography, TBTU=O-benzotriazol-1-yl-N,N,N′,N′-tetramethyl-uronium tetrafluoroborate, TEA=triethylamine, TFA=trifluoroacetic acid, THF=tetrahydrofuran, T₃P=propylphosphonic anhydride.

Compounds of formula (I), wherein R¹, R², R³, X, L, A, m and n are as defined herein may be synthesized according to the general procedure outlined in Scheme 1.

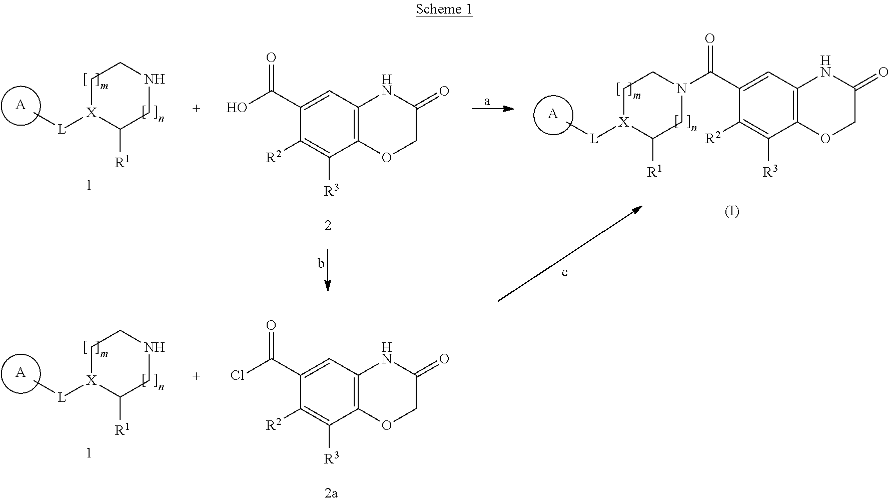

Reaction of intermediates 1 (either commercially available, or prepared by the methods described in Schemes 5 and 6 or in literature), wherein A, L, X, R¹, m and n are as defined herein, with benzoxazin-3(4H)-one carboxylic acid compounds 2, wherein R² and R³ are as defined herein, gives compounds of formula (I) (step a, Scheme 1). Amide couplings of this type can be accomplished by using one of the well-known coupling reagents such as, DCC, HATU, EDCI, HOBt, TBTU, T3P, etc. and a base like Huenig's base, triethyl amine or DMAP in a suitable solvent solvent like N,N-dimethylformamide, DMA, DCM or dioxane, preferably between 0° C. and room temperature.

Alternatively, the benzoxazin-3(4H)-one carboxylic acid compounds 2 can be converted into their acid chlorides 2a by treatment with e.g., thionyl chloride or oxalyl chloride, neat or optionally in a solvent such as DCM (Scheme 1, step b). Reaction of the acid chloride 2a with intermediates 1 in an appropriate solvent such as DCM or DMF and a base, e.g. Et₃N, Huenig's base, pyridine or DMAP at temperatures ranging from 0° C. to the reflux temperature of the solvent yields compounds of formula (I) (Scheme 1, step c).

In one embodiment, the compound of formula (I) is a compound of formula (Ia), wherein A, m, n, R¹, R² and R³ are as defined herein in relation to compounds of formula (I) and L is selected from the group consisting of heterocyclyl, —CHR⁶— and —CH₂CH₂—, preferably from —CHR⁶— and —CH₂CH₂—, wherein R⁶ is selected from the group consisting of hydrogen, alkyl, haloalkyl, cycloalkyl, aryl and heteroaryl, preferably from hydrogen and alkyl, in particular wherein R⁶ is hydrogen. Said compounds of formula (Ia) may be synthesized according to the general procedure outlined in Scheme 2.

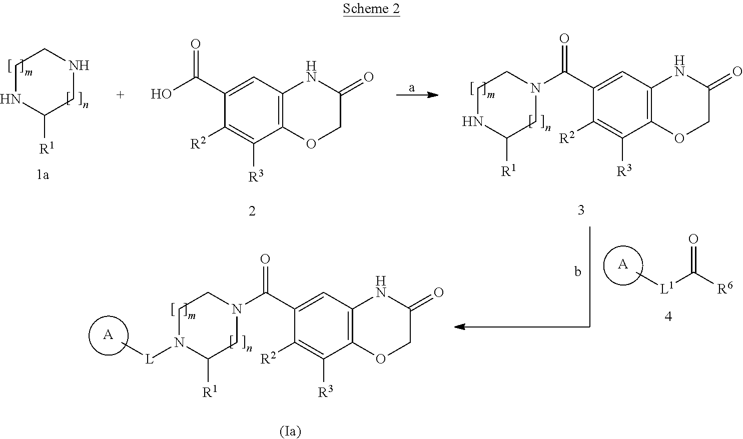

Thus, reaction of intermediates 1a (either commercially available or described in literature), wherein R¹, m and n are as defined herein, with benzoxazin-3(4H)-one carboxylic acid compounds 2, wherein R² and R³ are as defined herein, gives intermediates 3 (step a, Scheme 2), wherein R¹, R², R³, m and n are as defined herein. Subsequent reductive amination using intermediates 3 along with aldehydes or ketones 4 (either commercially available or described in literature), wherein A is as defined herein, L¹ is heterocyclyl, a covalent bond or methylene (CH₂), preferably a covalent bond or methylene and R⁶ is selected from the group consisting of hydrogen, alkyl, haloalkyl, cycloalkyl, aryl and heteroaryl, preferably from hydrogen and alkyl, in particular wherein R⁶ is hydrogen, in the presence of reducing agents such as sodium triacetoxyborohydride and optionally in the presence of a catalytic amount of acetic acid (i.e., less than 1 molar equivalent with respect to 4) in a solvent like DCE, MeOH, EtOH, THF, DCM or a mixture thereof, gives compounds of formula (Ia) (step d, Scheme 2).

Benzoxazin-3(4H)-one carboxylic acid compounds 2 can be prepared by a variety of conditions, which may be exemplified by the general synthetic procedures outlined in Schemes 3 and 4.

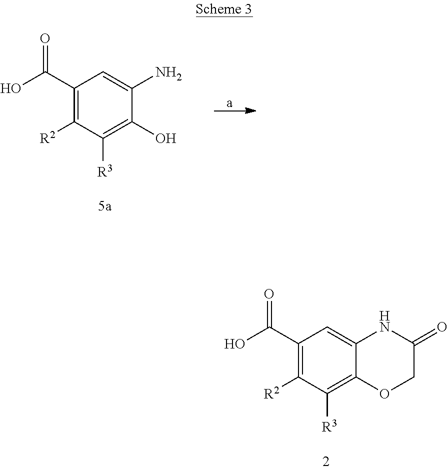

Cyclisation of commercially available 3-amino-4-hydroxy-benzoic acids 5a, wherein R² and R³ are as defined herein, can be achieved in presence of chloroacetyl chloride in a solvent like CHCl₃, DCM, THF or a mixture thereof, preferably in a mixture of THF and water and in a temperature range preferably between 0° C. and room temperature, to give the corresponding benzoxazin-3(4H)-one carboxylic acid compounds 2 (Scheme 3, step a).

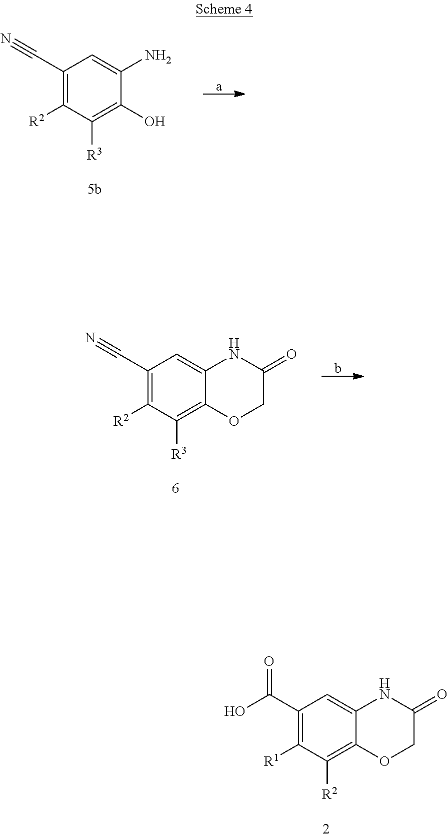

Alternatively, starting from commercially available 3-amino-4-hydroxy-benzonitriles 5b, wherein R² and R³ are as defined herein, reaction with chloroacetyl chloride in a solvent like CHCl₃, DCM or THF, preferably in a mixture of CHCl₃ and water, in the presence of a base such as Na₂CO₃, TEA, NaHCO₃, K₂CO₃, or a mixture thereof, preferably Na₂CO₃ and a phase transfer catalyst such as tetrabutylammonium chloride, tetrabutylammonium bromide, benzyltriethylammonium chloride or a mixture thereof, preferably benzyltriethylammonium chloride and in a temperature range between 0° C. and room temperature gives the corresponding benzoxazin-3(4H)-one carbonitrile compounds 6, wherein R² and R³ are as defined herein (Scheme 4, step a). Subsequent nitrile hydrolysis under alkaline conditions using a sodium hydroxide solution or under acidic conditions using a hydrochloric acid solution, preferably an aqueous concentrated hydrochloric acid solution (ca. 37% wt/wt in water) and in a temperature range between 70° C. to 100° C., preferably around boiling point of the reaction mixture, gives the corresponding benzoxazin-3(4H)-one carboxylic acid compounds 2 (Scheme 4, step b).

In one embodiment, intermediate 1 is an intermediate of type B or C, wherein A and n are as defined herein. Intermediates of type B can be prepared e.g., as exemplified by the synthetic procedures outlined in Scheme 5.

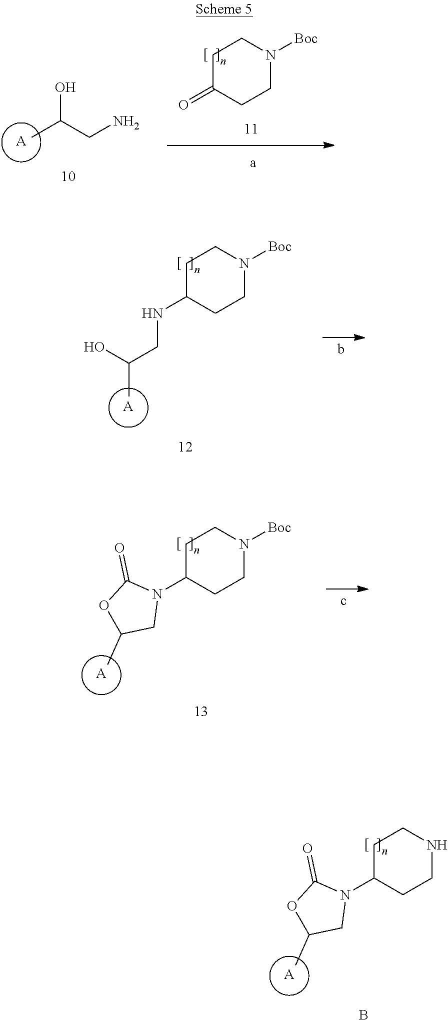

Intermediates of type C can be prepared e.g., as exemplified by the synthetic procedures outlined in Scheme 6.

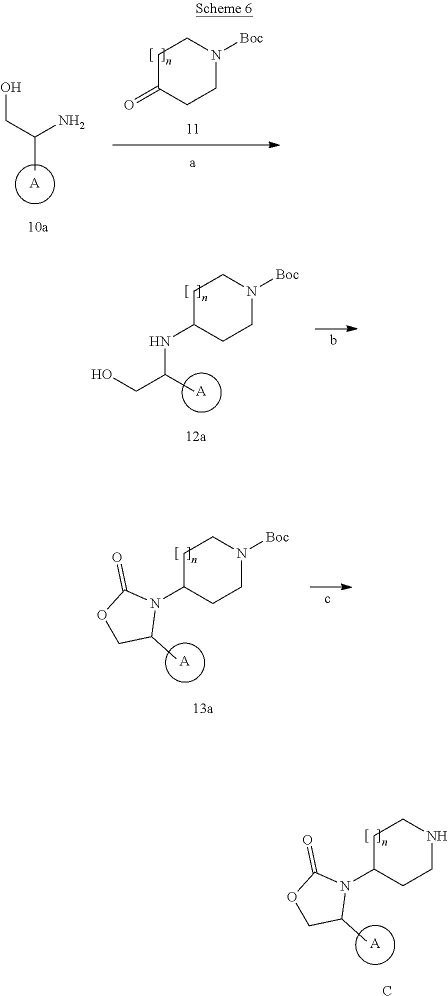

Reductive amination by reaction of amino alcohol compounds 10 or 10a, respectively, wherein A is as defined herein, preferably wherein A is aryl or heteroaryl substituted with R⁴ and R⁵ as defined herein, most preferably phenyl substituted with R⁴ and R⁵ as defined herein, and ketone 11, wherein n is as defined herein, in the presence of a reducing agent, such as sodium triacetoxyborohydride, sodium borohydride or sodium cyanoborohydride, preferably sodium triacetoxyborohydride and optionally in the presence of acetic acid, preferably in a catalytic amount (i.e., less than 1 molar equivalent with respect to 11) in a solvent like DCE, MeOH, EtOH, THF, DCM or a mixture thereof, preferably in DCE and in a temperature range between 0° C. and the boiling point of the solvent, preferably at room temperature, gives intermediates 12 or 12a, respectively (step a in Schemes 5 and 6). Subsequent cyclisation using e.g., CDI or triphosgene, preferably CDI, in a solvent like DCM, CH₃CN, THF, dioxane or a mixture thereof and in a temperature range between 0° C. and the boiling point of the solvent, preferably at room temperature, yields oxazolidone intermediates 13 or 13a, respectively (step b, Schemes 5 and 6). Finally, removal of the Boc protecting group using acidic conditions such as treatment with 4M HCl in dioxane or TFA in DCM, preferably with 4M HCl in dioxane, in a solvent like MeOH at around room temperature gives the corresponding intermediates of type B or C, respectively (step c, Schemes 5 and 6).

In one aspect, the present invention provides a process of manufacturing the compounds of formula (I) as described herein, comprising the steps of:

a) reacting an amine 1, wherein A, L, X, R¹, m and n are as described herein,

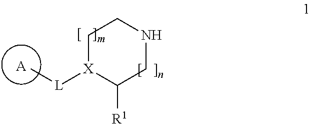

with an acid 2, wherein R² and R³ are as described herein

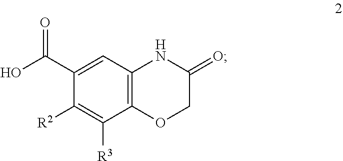

or

b) reacting an amine 1, wherein A, L, X, R¹, m and n are as described herein,

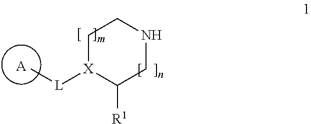

with an acid chloride 2a, wherein R² and R³ are as described herein

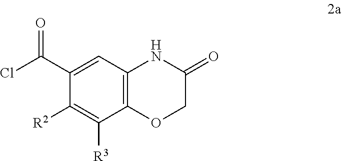

to form said compound of formula (I).

In one embodiment, there is provided a process of manufacturing compounds of formula (I) as described herein, comprising reacting an amine 1, wherein A, L, X, R¹, m and n are as described herein,

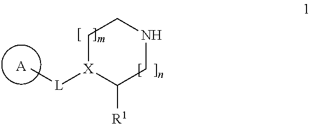

with an acid 2, wherein R² and R³ are as described herein

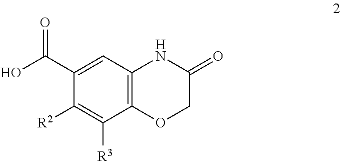

to form said compound of formula (I).

In one embodiment, there is provided a process of manufacturing compounds of formula (I) as described herein, comprising reacting an amine 1, wherein A, L, X, R¹, m and n are as described herein,

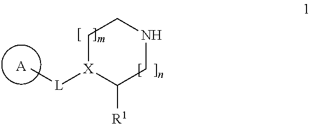

with an acid chloride 2a, wherein R² and R³ are as described herein

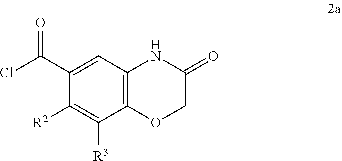

to form said compound of formula (I).

In a further aspect, there is provided a process of manufacturing compounds of formula (I), wherein said compounds of formula (I) are compounds of formula (Ia) as described herein, comprising reacting an amine 3, wherein R¹, R², R³, m and n are as defined herein,

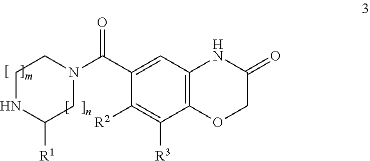

with a compound of formula 4, wherein A, L¹ and R⁶ are as defined herein,

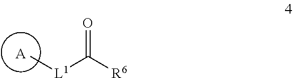

in the presence of a reducing agent, preferably in the presence of sodium triacetoxyborohydride, to form said compounds of formula (Ia).

In one aspect, the present invention provides a compound of formula (I) as described herein, when manufactured according to any one of the processes described herein.

### MAGL Inhibitory Activity

Compounds of the present invention are MAGL inhibitors. Thus, in one aspect, the present invention provides the use of compounds of formula (I) as described herein, or a pharmaceutically acceptable salt thereof, for inhibiting MAGL in a mammal.

In a further aspect, the present invention provides compounds of formula (I) as described herein, or a pharmaceutically acceptable salt thereof, for use in a method of inhibiting MAGL in a mammal.

In a further aspect, the present invention provides the use of compounds of formula (I) as described herein, or a pharmaceutically acceptable salt thereof, for the preparation of a medicament for inhibiting MAGL in a mammal.

In a further aspect, the present invention provides a method for inhibiting MAGL in a mammal, which method comprises administering an effective amount of a compound of formula (I) as described herein to the mammal.

Compounds were profiled for MAGL inhibitory activity by measuring the enzymatic activity of MAGL by following the hydrolysis of 4-nitorphenylacetate resulting in 4-nitrophenol, which absorbs at 405-412 nm (G. G. Muccioli, G. Labar, D. M. Lambert, Chem. Bio. Chem. 2008, 9, 2704-2710).

The assay was carried out in 384 well assay plates (black with clear bottom, non-binding surface treated, Corning Ref. 3655) in a total volume of 40 μL. Compound dilutions were made in 100% DMSO (VWR Chemicals 23500.297) in a polypropylene plate in 3-fold dilution steps to give a final concentration range in the assay from 25 μM to 1.7 nM. 1 μL compound dilutions (100% DMSO) were added to 19 μL MAGL (recombinant wild-type) in assay buffer (50 mM TRIS (GIBCO, 15567-027), 1 mM EDTA (Fluka, 03690-100 ml)). The plate was shaked for 1 min at 2000 rpm (Variomag Teleshake) and then incubated for 15 min at RT. To start the reaction, 20 μL 4-Nitrophenlyacetate (Sigma N-8130) in assay buffer with 6% EtOH was added. The final concentrations in the assay were 1 nM MAGL and 300 μM 4-Nitrophenylacetate. After shaking (1 m, 2000 rpm) and 5 min incubation at RT, the absorbance at 405 nm was measured for a first time (Molecular Devices, SpectraMax Paradigm). A second measurement was then done after incubation for 80 min at RT. From the two measurements, the slope was calculated by subtracting the first from the second measurement.

TABLE 1

| | | | IC₅₀ MAGL |
|---|---|---|---|
| Example | Name | Structure | [μM] |
| 1 | 6-(4-benzylpiperidine-1- carbonyl)-4H-1,4- benzoxazin-3-one | 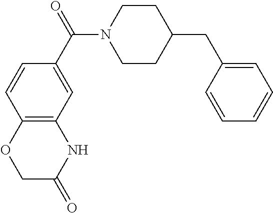 [SMILES: O=C1COc2ccc(C(=O)N3CCC(Cc4ccccc4)CC3)cc2N1] | 0.10050 |
| 2 | 6-(4-benzylpiperazine-1- carbonyl)-4H-1,4- benzoxazin-3-one | 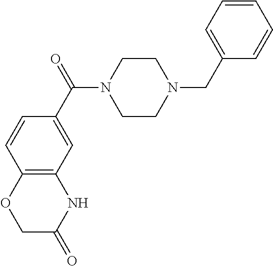 [SMILES: O=C1COc2ccc(C(=O)N3CCN(Cc4ccccc4)CC3)cc2N1] | 3.72000 |
| 3 | 6-[4-[(4- fluorophenyl)methyl] piperidine-1-carbonyl]-4H-1,4- benzoxazin-3-one | 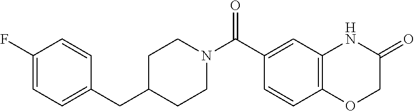 [SMILES: O=C1COc2ccc(C(=O)N3CCC(Cc4ccc(F)cc4)CC3)cc2N1] | 0.08800 |
| 4 | 6-[4-[[4- (trifluoromethyl)phenyl] methyl]piperidine-1-carbonyl]- 4H-1,4-benzoxazin-3-one | 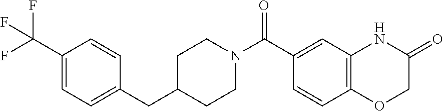 [SMILES: O=C1COc2ccc(C(=O)N3CCC(Cc4ccc(C(F)(F)F)cc4)CC3)cc2N1] | 0.03467 |
| 5 | 6-[4-[(4- methoxyphenyl)methyl] piperidine-1-carbonyl]-4H-[1,4- benzoxazin-3-one | 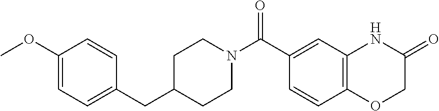 [SMILES: COc1ccc(CC2CCN(C(=O)c3ccc4c(c3)NC(=O)CO4)CC2)cc1] | 0.17000 |
| 6 | 6-[4-[(3- methoxyphenyl)methyl] piperidine-1-carbonyl]-4H-1,4- benzoxazin-3-one | 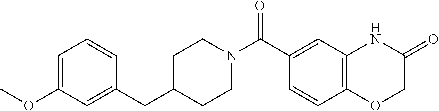 [SMILES: COc1cccc(CC2CCN(C(=O)c3ccc4c(c3)NC(=O)CO4)CC2)c1] | 0.05200 |
| 7 | 6-(4-benzyl-4- hydroxypiperidine-1- carbonyl)-4H-1,4- benzoxazin-3-one | 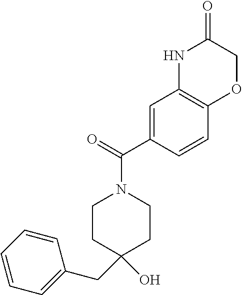 [SMILES: O=C1COc2ccc(C(=O)N3CCC(O)(Cc4ccccc4)CC3)cc2N1] | 4.35000 |
| 8 | 6-[4-[(3- fluorophenyl)methyl] piperidine-1-carbonyl]-4H-1,4- benzoxazin-3-one | 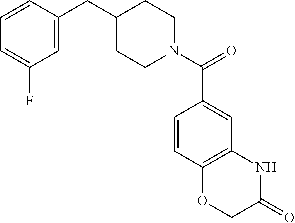 [SMILES: O=C1COc2ccc(C(=O)N3CCC(Cc4cccc(F)c4)CC3)cc2N1] | 0.04700 |
| 9 | 6-[4-[(3,5- difluorophenyl)methyl] piperidine-1-carbonyl]-4H-1,4- benzoxazin-3-one | 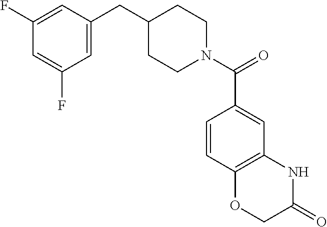 [SMILES: O=C1COc2ccc(C(=O)N3CCC(Cc4cc(F)cc(F)c4)CC3)cc2N1] | 0.05800 |
| 10 | 6-[4- (benzenesulfonyl)piperidine- 1-carbonyl]-4H-1,4- benzoxazin-3-one | 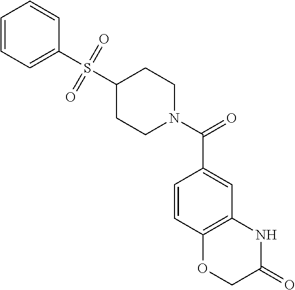 [SMILES: O=C1COc2ccc(C(=O)N3CCC(S(=O)(=O)c4ccccc4)CC3)cc2N1] | 4.22000 |
| 11 | 6-[4-[(5S)-2-oxo-5-phenyl- 1,3-oxazolidin-3- yl]piperidine-1-carbonyl]- 4H-1,4-benzoxazin-3-one | 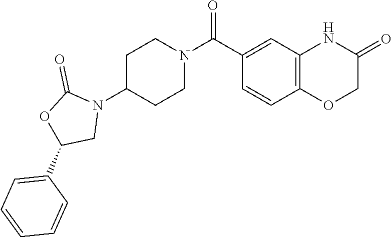 [SMILES: O=C1COc2ccc(C(=O)N3CCC(N4C[C@H](c5ccccc5)OC4=O)CC3)cc2N1] | 1.26000 |
| 12 | 6-[4-[(4- chlorophenyl)methyl] piperidine-1-carbonyl]-4H-1,4- benzoxazin-3-one | 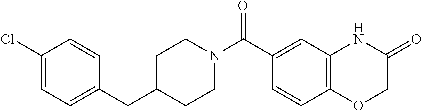 [SMILES: O=C1COc2ccc(C(=O)N3CCC(Cc4ccc(Cl)cc4)CC3)cc2N1] | 0.04800 |
| 13 | 6-(4-benzoylpiperidine-1- carbonyl)-4H-1,4- benzoxazin-3-one | 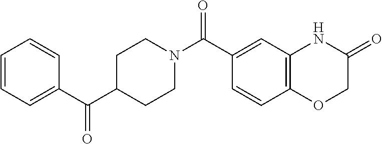 [SMILES: O=C1COc2ccc(C(=O)N3CCC(C(=O)c4ccccc4)CC3)cc2N1] | 0.32000 |
| 14 | 6-(4-phenoxypiperidine-1- carbonyl)-4H-1,4- benzoxazin-3-one | 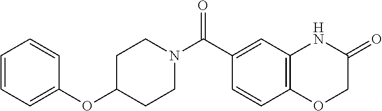 [SMILES: O=C1COc2ccc(C(=O)N3CCC(Oc4ccccc4)CC3)cc2N1] | 0.99000 |
| 15 | 6-[4-[4- (trifluoromethyl)phenoxy] piperidine-1-carbonyl]-4H-1,4- benzoxazin-3-one | 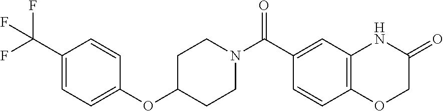 [SMILES: O=C1COc2ccc(C(=O)N3CCC(Oc4ccc(C(F)(F)F)cc4)CC3)cc2N1] | 0.07600 |
| 16 | 6-[4-[(2,3- difluorophenyl)methyl] piperidine-1-carbonyl]-4H-1,4- benzoxazin-3-one | 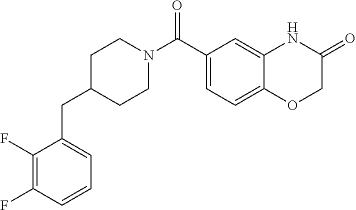 [SMILES: O=C1COc2ccc(C(=O)N3CCC(Cc4cccc(F)c4F)CC3)cc2N1] | 0.06600 |
| 17 | 6-[4-[[3- (trifluoromethyl)phenyl] methyl]piperidine-1-carbonyl]- 4H-1,4-benzoxazin-3-one | 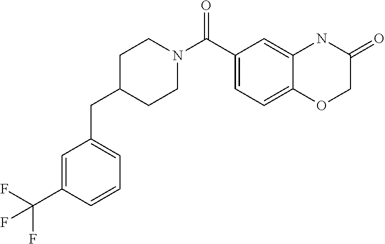 [SMILES: O=C1COc2ccc(C(=O)N3CCC(Cc4cccc(C(F)(F)F)c4)CC3)cc2N1] | 0.03950 |
| 18 | 6-(3-phenoxypyrrolidine-1- carbonyl)-4H-1,4- benzoxazin-3-one | 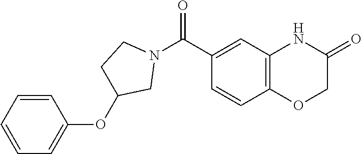 [SMILES: O=C1COc2ccc(C(=O)N3CCC(Oc4ccccc4)C3)cc2N1] | 1.04000 |
| 19 | 6-[4-[(5R)-2-oxo-5-phenyl- 1,3-oxazolidin-3- yl]piperidine-1-carbonyl]- 4H-1,4-benzoxazin-3-one | 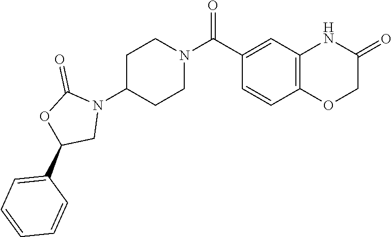 [SMILES: O=C1COc2ccc(C(=O)N3CCC(N4C[C@@H](c5ccccc5)OC4=O)CC3)cc2N1] | 2.49000 |
| 20 | 4-[[1-(3-oxo-4H-1,4- benzoxazine-6- carbonyl)piperidin-4- yl]methyl]benzonitrile | 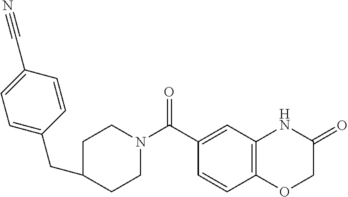 [SMILES: N#Cc1ccc(CC2CCN(C(=O)c3ccc4c(c3)NC(=O)CO4)CC2)cc1] | 0.37000 |
| 21 | 6-[4-[(2- phenylphenyl)methyl] piperidine-1-carbonyl]-4H-1,4- benzoxazin-3-one | 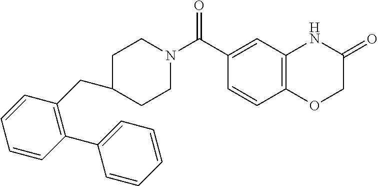 [SMILES: O=C1COc2ccc(C(=O)N3CCC(Cc4ccccc4-c4ccccc4)CC3)cc2N1] | 0.13000 |
| 22 | 6-[4-(pyridin-2- ylmethyl)piperidine-1- carbonyl]-4H-1,4- benzoxazin-3-one | 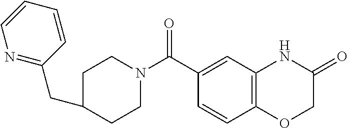 [SMILES: O=C1COc2ccc(C(=O)N3CCC(Cc4ccccn4)CC3)cc2N1] | 1.70000 |
| 23 | 6-[4-[(4- chlorophenyl)methyl]-4- fluoropiperidine-1-carbonyl]- 4H-1,4-benzoxazin-3-one | 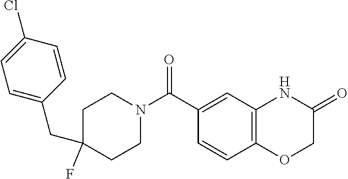 [SMILES: O=C1COc2ccc(C(=O)N3CCC(F)(Cc4ccc(Cl)cc4)CC3)cc2N1] | 0.30000 |
| 24 | 6-(4-benzylpiperidine-1- carbonyl)-8-fluoro-4H-1,4- benzoxazin-3-one | 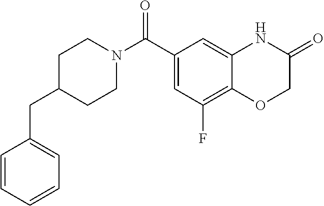 [SMILES: O=C1COc2c(F)cc(C(=O)N3CCC(Cc4ccccc4)CC3)cc2N1] | 0.14000 |
| 25 | N-methyl-N-[1-(3-oxo-4H- 1,4-benzoxazine-6- carbonyl)piperidin-4- yl]benzamide | 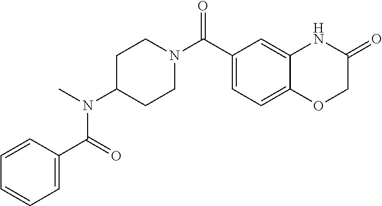 [SMILES: CN(C(=O)c1ccccc1)C1CCN(C(=O)c2ccc3c(c2)NC(=O)CO3)CC1] | 0.83000 |
| 26 | 6-(4-benzylpiperidine-1- carbonyl)-4H-pyrido[3,2- b][1,4]oxazin-3-one | 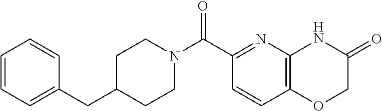 [SMILES: O=C1COc2ccc(C(=O)N3CCC(Cc4ccccc4)CC3)nc2N1] | 2.06000 |
| 27 | 6-[4-(piperidine-1- carbonyl)piperidine-1- carbonyl]-4H-1,4- benzoxazin-3-one | 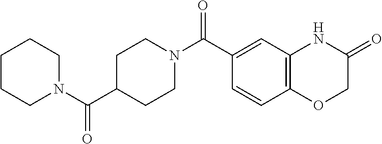 [SMILES: O=C1COc2ccc(C(=O)N3CCC(C(=O)N4CCCCC4)CC3)cc2N1] | 4.24000 |
| 28 | 6-(3-benzylpyrrolidine-1- carbonyl)-4H-1,4- benzoxazin-3-one | 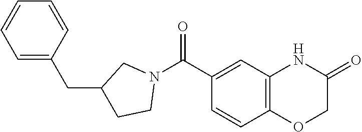 [SMILES: O=C1COc2ccc(C(=O)N3CCC(Cc4ccccc4)C3)cc2N1] | 0.84000 |
| 29 | 6-[3-[[4- (trifluoromethyl)phenyl] methyl]azetidine-1-carbonyl]- 4H-1,4-benzoxazin-3-one | 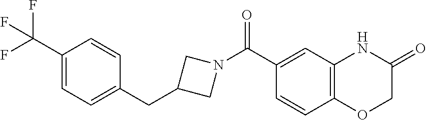 [SMILES: O=C1COc2ccc(C(=O)N3CC(Cc4ccc(C(F)(F)F)cc4)C3)cc2N1] | 0.15000 |
| 30 | 6-[3-[[3- (trifluoromethyl)phenyl] methyl]pyrrolidine-1- carbonyl]-4H-1,4-benzoxazin- 3-one | 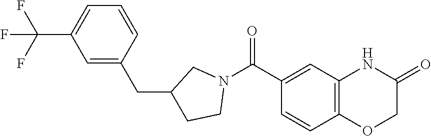 [SMILES: O=C1COc2ccc(C(=O)N3CCC(Cc4cccc(C(F)(F)F)c4)C3)cc2N1] | 0.22000 |
| 31 | 6-[3-[[4- (trifluoromethyl)phenyl] methyl]pyrrolidine-1- carbonyl]-4H-1,4-benzoxazin- 3-one | 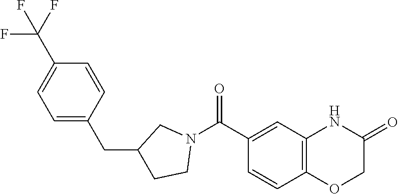 [SMILES: O=C1COc2ccc(C(=O)N3CCC(Cc4ccc(C(F)(F)F)cc4)C3)cc2N1] | 0.40000 |
| 32 | 6-[4-[4- (trifluoromethyl)benzoyl] piperazine-1-carbonyl]-4H-1,4- benzoxazin-3-one | 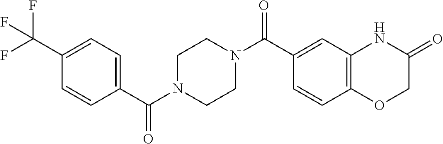 [SMILES: O=C1COc2ccc(C(=O)N3CCN(C(=O)c4ccc(C(F)(F)F)cc4)CC3)cc2N1] | 2.18000 |
| 33 | N-cyclopropyl-N-[1-(3-oxo- 4H-1,4-benzoxazine-6- carbonyl)piperidin-4-yl]-2- phenylacetamide | 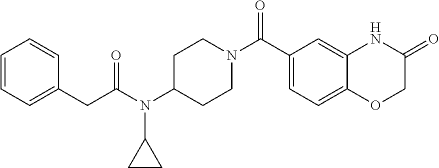 [SMILES: O=C1COc2ccc(C(=O)N3CCC(N(C(=O)Cc4ccccc4)C4CC4)CC3)cc2N1] | 0.31000 |
| 34 | 6-[3-methyl-4-[[4- (trifluoromethyl)phenyl] methyl]piperazine-1-carbonyl]- 4H-1,4-benzoxazin-3-one | 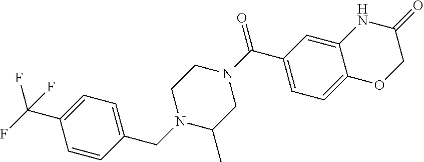 [SMILES: CC1CN(C(=O)c2ccc3c(c2)NC(=O)CO3)CCN1Cc1ccc(C(F)(F)F)cc1] | 0.44925 |
| 35 | 6-[4-[2-(3- chlorophenyl)ethyl]-3-(1H- pyrazol-3-yl)piperazine-1- carbonyl]-4H-1,4- benzoxazin-3-one | 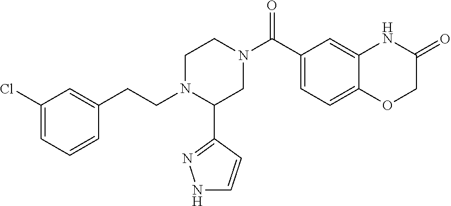 [SMILES: O=C1COc2ccc(C(=O)N3CCN(CCc4cccc(Cl)c4)C(c4cc[nH]n4)C3)cc2N1] | 1.82700 |
| 36 | 6-[3-(trifluoromethyl)-4-[[4- (trifluoromethyl)phenyl] methyl]piperazine-1-carbonyl]- 4H-1,4-benzoxazin-3-one | 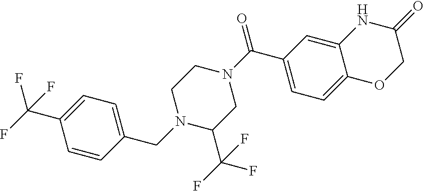 [SMILES: O=C1COc2ccc(C(=O)N3CCN(Cc4ccc(C(F)(F)F)cc4)C(C(F)(F)F)C3)cc2N1] | 0.11160 |
| 37 | 6-[3-(1H-pyrazol-3-yl)-4-[[4- (trifluoromethyl)phenyl] methyl]piperazine-1-carbonyl]- 4H-1,4-benzoxazin-3-one | 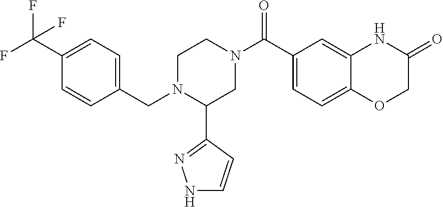 [SMILES: O=C1COc2ccc(C(=O)N3CCN(Cc4ccc(C(F)(F)F)cc4)C(c4cc[nH]n4)C3)cc2N1] | 2.24965 |

In one aspect, the present invention provides compounds of formula (I) and their pharmaceutically acceptable salts as described herein, wherein said compounds of formula (I) and their pharmaceutically acceptable salts have IC₅₀'s for MAGL inhibition below 25 μM, preferably below 10 μM, more preferably below 5 μM as measured in the MAGL assay described herein.

In one embodiment, compounds of formula (I) and their pharmaceutically acceptable salts as described herein have IC₅₀ (MAGL inhibition) values between 0.000001 μM and 25 μM, particular compounds have IC₅₀ values between 0.000005 μM and 10 μM, further particular compounds have IC₅₀ values between 0.00005 μM and 5 μM, as measured in the MAGL assay described herein.

In one embodiment, the present invention provides compounds of formula (I) and their pharmaceutically acceptable salts as described herein, wherein said compounds of formula (I) and their pharmaceutically acceptable salts have an IC₅₀ for MAGL below 25 μM, preferably below 10 μM, more preferably below 5 μM as measured in an assay comprising the steps of
a) providing a solution of a compound formula (I), or a pharmaceutically acceptable salt thereof, in DMSO;
b) providing a solution of MAGL (recombinant wild-type) in assay buffer (50 mM tris(hydroxymethyl)aminomethane; 1 mM ethylenediaminetetraacetic acid);
c) adding 1 μL of compound solution from step a) to 19 μL of MAGL solution from step b);
d) shaking the mixture for 1 min at 2000 rpm;
e) incubating for 15 min at RT;
f) adding 20 μL of a solution of 4-nitrophenlyacetate in assay buffer (50 mM tris(hydroxymethyl)aminomethane; 1 mM ethylenediaminetetraacetic acid, 6% EtOH);
g) shaking the mixture for 1 min at 2000 rpm;
h) incubating for 5 min at RT;
i) measuring the absorbance of the mixture at 405 nm a first time;
j) incubating a further 80 min at RT;
k) measuring the absorbance of the mixture at 405 nm a second time;
l) substracting the absorbance measured under i) from the absorbance measured under k) and calculating the slope of absorbance;
wherein:
i) the concentration of the compound of formula (I), or the pharmaceutically acceptable salt thereof in the assay after step f) is in the range of 25 μM to 1.7 nM;
ii) the concentration of MAGL in the assay after step f) is 1 nM;
iii) the concentration of 4-nitrophenylacetate in the assay after step f) is 300 μM; and
iv) steps a) to 1) are repeated for at least 3 times, each time with a different concentration of the compound of formula (I), or the pharmaceutically acceptable salt thereof.

### Using the Compounds of the Invention

In one aspect, the present invention provides compounds of formula (I) as described herein, or a pharmaceutically acceptable salt thereof, for use as therapeutically active substance.

In a further aspect, the present invention provides the use of compounds of formula (I) as described herein, or a pharmaceutically acceptable salt thereof, for the treatment or prophylaxis of neuroinflammation, neurodegenerative diseases, pain, cancer or mental disorders, or any possible combination thereof, in a mammal.

In one embodiment, the present invention provides the use of compounds of formula (I) as described herein, or a pharmaceutically acceptable salt thereof, for the treatment or prophylaxis of neuroinflammation or neurodegenerative diseases, or any possible combination thereof, in a mammal.

In one embodiment, the present invention provides the use of compounds of formula (I) as described herein, or a pharmaceutically acceptable salt thereof, for the treatment or prophylaxis of neurodegenerative diseases in a mammal.

In one embodiment, the present invention provides the use of compounds of formula (I) as described herein, or a pharmaceutically acceptable salt thereof, for the treatment or prophylaxis of cancer in a mammal.

In one aspect, the present invention provides the use of compounds of formula (I) as described herein, or a pharmaceutically acceptable salt thereof, for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, cancer or pain, or any possible combination thereof, in a mammal.

In a preferred embodiment, the present invention provides the use of compounds of formula (I) as described herein, or a pharmaceutically acceptable salt thereof, for the treatment or prophylaxis of multiple sclerosis, Alzheimer's disease or Parkinson's disease, or any possible combination thereof, in a mammal.

In a particularly preferred embodiment, the present invention provides the use of compounds of formula (I) as described herein, or a pharmaceutically acceptable salt thereof, for the treatment or prophylaxis of multiple sclerosis in a mammal.

In one aspect, the present invention provides compounds of formula (I) as described herein, or a pharmaceutically acceptable salt thereof, for use in the treatment or prophylaxis of neuroinflammation, neurodegenerative diseases, pain, cancer or mental disorders, or any possible combination thereof, in a mammal.

In one embodiment, the present invention provides compounds of formula (I) as described herein, or a pharmaceutically acceptable salt thereof, for use in the treatment or prophylaxis of neuroinflammation or neurodegenerative diseases, or any possible combination thereof, in a mammal.

In one embodiment, the present invention provides compounds of formula (I) as described herein, or a pharmaceutically acceptable salt thereof, for use in the treatment or prophylaxis of cancer in a mammal.

In one embodiment, the present invention provides compounds of formula (I) as described herein, or a pharmaceutically acceptable salt thereof, for use in the treatment or prophylaxis of neurodegenerative diseases in a mammal.

In one aspect, the present invention provides compounds of formula (I) as described herein, or a pharmaceutically acceptable salt thereof, for use in the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, cancer or pain, or any possible combination thereof, in a mammal.

In a preferred embodiment, the present invention provides compounds of formula (I) as described herein, or a pharmaceutically acceptable salt thereof, for use in the treatment or prophylaxis of multiple sclerosis, Alzheimer's disease or Parkinson's disease, or any possible combination thereof, in a mammal.

In a particularly preferred embodiment, the present invention provides compounds of formula (I) as described herein, or a pharmaceutically acceptable salt thereof, for use in the treatment or prophylaxis of multiple sclerosis in a mammal.

In one aspect, the present invention provides the use of compounds of formula (I) as described herein, or a pharmaceutically acceptable salt thereof, for the preparation of a medicament for the treatment or prophylaxis of neuroinflammation, neurodegenerative diseases, pain, cancer or mental disorders, or any possible combination thereof, in a mammal.

In one embodiment, the present invention provides the use of compounds of formula (I) as described herein, or a pharmaceutically acceptable salt thereof, for the preparation of a medicament for the treatment or prophylaxis of neuroinflammation or neurodegenerative diseases, or any possible combination thereof, in a mammal.

In one embodiment, the present invention provides the use of compounds of formula (I) as described herein, or a pharmaceutically acceptable salt thereof, for the preparation of a medicament for the treatment or prophylaxis of neurodegenerative diseases in a mammal.

In one embodiment, the present invention provides the use of compounds of formula (I) as described herein, or a pharmaceutically acceptable salt thereof, for the preparation of a medicament for the treatment or prophylaxis of cancer in a mammal.

In a further aspect, the present invention provides the use of compounds of formula (I) as described herein, or a pharmaceutically acceptable salt thereof, for the preparation of a medicament for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression, cancer or pain, or any possible combination thereof, in a mammal.

In a preferred embodiment, the present invention provides the use of compounds of formula (I) as described herein, or a pharmaceutically acceptable salt thereof, for the preparation of a medicament for the treatment or prophylaxis of multiple sclerosis, Alzheimer's disease or Parkinson's disease, or any possible combination thereof, in a mammal.

In a particularly preferred embodiment, the present invention provides the use of compounds of formula (I) as described herein, or a pharmaceutically acceptable salt thereof, for the preparation of a medicament for the treatment or prophylaxis of multiple sclerosis in a mammal.

In one aspect, the present invention provides a method for the treatment or prophylaxis of neuroinflammation, neurodegenerative diseases, pain, cancer or mental disorders, or any possible combination thereof, in a mammal, which method comprises administering an effective amount of a compound of formula (I) as described herein to the mammal.

In one embodiment, the present invention provides a method for the treatment or prophylaxis of neuroinflammation or neurodegenerative diseases, or any possible combination thereof, in a mammal, which method comprises administering an effective amount of a compound of formula (I) as described herein to the mammal.

In one embodiment, the present invention provides a method for the treatment or prophylaxis of neurodegenerative diseases in a mammal, which method comprises administering an effective amount of a compound of formula (I) as described herein to the mammal.

In one aspect, the present invention provides a method for the treatment or prophylaxis of multiple sclerosis, Alzheimer's disease, Parkinson's disease, amyotrophic lateral sclerosis, traumatic brain injury, neurotoxicity, stroke, epilepsy, anxiety, migraine, depression or pain, or any possible combination thereof, in a mammal, which method comprises administering an effective amount of a compound of formula (I) as described herein to the mammal.

In a preferred embodiment, the present invention provides a method for the treatment or prophylaxis of multiple sclerosis, Alzheimer's disease or Parkinson's disease, or any possible combination thereof, in a mammal, which method comprises administering an effective amount of a compound of formula (I) as described herein to the mammal.

In a particularly preferred embodiment, the present invention provides a method for the treatment or prophylaxis of multiple sclerosis in a mammal, which method comprises administering an effective amount of a compound of formula (I) as described herein to the mammal.

### Pharmaceutical Compositions and Administration

In one aspect, the present invention provides a pharmaceutical composition comprising a compound of formula (I) as described herein and a therapeutically inert carrier.

The compounds of formula (I) and their pharmaceutically acceptable salts can be used as medicaments (e.g. in the form of pharmaceutical preparations). The pharmaceutical preparations can be administered internally, such as orally (e.g. in the form of tablets, coated tablets, dragees, hard and soft gelatin capsules, solutions, emulsions or suspensions), nasally (e.g. in the form of nasal sprays) or rectally (e.g. in the form of suppositories). However, the administration can also be effected parentally, such as intramuscularly or intravenously (e.g. in the form of injection solutions).

The compounds of formula (I) and their pharmaceutically acceptable salts can be processed with pharmaceutically inert, inorganic or organic adjuvants for the production of tablets, coated tablets, dragees and hard gelatin capsules. Lactose, corn starch or derivatives thereof, talc, stearic acid or its salts etc. can be used, for example, as such adjuvants for tablets, dragees and hard gelatin capsules.

Suitable adjuvants for soft gelatin capsules are, for example, vegetable oils, waxes, fats, semi-solid substances and liquid polyols, etc.

Suitable adjuvants for the production of solutions and syrups are, for example, water, polyols, saccharose, invert sugar, glucose, etc.

Suitable adjuvants for injection solutions are, for example, water, alcohols, polyols, glycerol, vegetable oils, etc.

Suitable adjuvants for suppositories are, for example, natural or hardened oils, waxes, fats, semi-solid or liquid polyols, etc.

Moreover, the pharmaceutical preparations can contain preservatives, solubilizers, viscosity-increasing substances, stabilizers, wetting agents, emulsifiers, sweeteners, colorants, flavorants, salts for varying the osmotic pressure, buffers, masking agents or antioxidants. They can also contain still other therapeutically valuable substances.

The dosage can vary in wide limits and will, of course, be fitted to the individual requirements in each particular case. In general, in the case of oral administration a daily dosage of about 0.1 mg to 20 mg per kg body weight, preferably about 0.5 mg to 4 mg per kg body weight (e.g. about 300 mg per person), divided into preferably 1-3 individual doses, which can consist, for example, of the same amounts, should be appropriate. It will, however, be clear that the upper limit given herein can be exceeded when this is shown to be indicated.

In accordance with the invention, the compounds of formula (I) or their pharmaceutically acceptable salts can be used for the treatment or prophylaxis of type 2 diabetes related microvascular complications (such as, but not limited to diabetic retinopathy, diabetic neuropathy and diabetic nephropathy), coronary artery disease, obesity and underlying inflammatory diseases, chronic inflammatory and autoimmune/inflammatory diseases.

### EXAMPLES

The invention will be more fully understood by reference to the following examples. The claims should not, however, be construed as limited to the scope of the examples.

In case the preparative examples are obtained as a mixture of enantiomers, the pure enantiomers can be separated by methods described herein or by methods known to the man skilled in the art, such as e.g., chiral chromatography (e.g., chiral SFC) or crystallization.

All reaction examples and intermediates were prepared under an argon atmosphere if not specified otherwise.

### Intermediate A-1

### 3-Oxo-4H-1,4-benzoxazine-6-carboxylic acid

To a solution of 3-amino-4-hydroxybenzoic acid (10.0 g, 65.3 mmol) and potassium carbonate (10.83 g, 78.36 mmol) in THF (15 mL) and water (30 mL) cooled at 0° C. was added chloroacetyl chloride (8.85 g, 78.36 mmol) and the reaction mixture was then stirred at 25° C. overnight. The reaction was quenched using concentrated hydrochloric acid until pH of 2. The solid precipitate was filtered off, washed with water (50 mL) and MeOH (5 mL) to give the crude title compound (8.6 g, 65%) as light yellow solid. MS (ESI): m/z=194.1 [M+H]⁺.

### Intermediate A-2

### 8-Fluoro-3-oxo-4H-1,4-benzoxazine-6-carboxylic acid

The title compound was prepared in analogy to intermediate A-1, but using 3-amino-5-fluoro-4-hydroxybenzoic acid (CAS RN 1025127-44-9) to give the title compound as an off-white solid. MS (ESI): m/z=212.1 [M+H]⁺.

### Intermediate B-1

### (5S)-5-Phenyl-3-(4-piperidyl)oxazolidin-2-one

### Step [A] tert-butyl 4-[[(2S)-2-hydroxy-2-phenyl-ethyl]amino]piperidine-1-carboxylate

To a solution of (S)-2-amino-1-phenylethanol (CAS RN 56613-81-1, 0.207 g, 1.51 mmol) and tert-butyl 4-oxopiperidine-1-carboxylate (CAS RN 79099-07-3, 0.250 g, 1.25 mmol) in DCE (2 mL) cooled at 0° C. with an ice bath was added sodium triacetoxyborohydride (0.399 g, 1.88 mmol) in 2 portions followed by acetic acid (0.014 mL, 0.251 mmol) and the resulting reaction mixture was stirred at room temperature over night. The mixture was diluted with EtOAc, poured into a sat. NaHCO₃ aq. solution and the aqueous layer was extracted with EtOAc. The combined organic layers were washed with brine, dried over Na₂SO₄, filtered and evaporated to dryness to give the crude title compound (0.42 g) as a colorless solid. MS (ESI): m/z=321.21 [M+H]⁺.

### Step [B] tert-butyl 4-[(5S)-2-oxo-5-phenyl-oxazolidin-3-yl]piperidine-1-carboxylate

To a solution of (R)-tert-butyl 4-((2-hydroxy-2-phenylethyl)amino)piperidine-1-carboxylate (0.42 g, 1.31 mmol) in dioxane (3 mL) was added CDI (0.101 g, 0.624 mmol) and the reaction mixture was stirred at room temperature over night. The mixture was concentrated in vacuo, diluted with EtOAc and washed with water. The combined organic layers were washed with brine, dried over Na₂SO₄, filtered and evaporated. The residue was purified by silica gel flash chromatography, eluting with a 0 to 50% EtOAc-heptane gradient to give the title compound (0.410 g, 90%) as a colorless solid; MS (ESI): m/z=291.2 [M-tBu+H]⁺.

### Step [C] (5S)-5-phenyl-3-(4-piperidyl)oxazolidin-2-one

4M HCl in dioxane (1.91 mL, 7.62 mmol) was added to a solution of tert-butyl 4-[(5S)-2-oxo-5-phenyl-oxazolidin-3-yl]piperidine-1-carboxylate (0.330 g, 0.953 mmol) in DCM (3 mL) and the reaction mixture was stirred at room temperature for 4 hours. The mixture was evaporated to dryness and the residue was triturated with diisopropylether. The solid precipitate was filtered off and further dried under the high vacuum to give the title compound (0.225 g, 96%) as a colorless solid as hydrochloride; MS (ESI): m/z=247.2 [M+H]⁺.

### Intermediate B-2

(5R)-5-Phenyl-3-(4-piperidyl)oxazolidin-2-one The title compound was prepared in analogy to intermediate B-1, but using in step [A] (R)-2-amino-1-phenylethanol (CAS RN 2549-14-6) to give the title compound as a colorless solid; MS (ESI): m/z=247.1 [M+H]⁺.

### Example 1

### 6-(4-Benzylpiperidine-1-carbonyl)-4H-1,4-benzoxazin-3-one

In a flask, 3-oxo-4H-1,4-benzoxazine-6-carboxylic acid (Intermediate A-1, 0.042 g, 0.217 mmol, 4-benzylpiperidine (CAS RN 31252-42-3, 0.038 mL, 0.217 mmol) and HATU (0.091 g, 0.239 mmol) were mixed in DMF (1 mL). Then, Hunig's base (0.095 mL, 0.544 mmol) was added and the reaction mixture was stirred at room temperature for 2 hours. The reaction was diluted with EtOAc, poured into water and the aqueous layer was extracted with EtOAc. The combined organic layers were washed with brine, dried over Na₂SO₄, filtered and concentrated in vacuo. The residue was purified by silica gel flash chromatography eluting with a 0 to 100% EtOAc-heptane gradient to give the title compound (0.050 g, 66%) as a colorless solid; MS (ESI): m/z=351.2 [M+H]⁺.

The following examples listed in Table 2 were prepared in analogy to the procedures described for the preparation of example 1 by using the indicated intermediates and/or commercial compounds and using the mentioned purification method such as preparative HPLC (Gemini NX column) or silica gel flash chromatography.

TABLE 2

| | | | MS, m/z |
|---|---|---|---|
| | Name | | [M + H]⁺ |
| | Aspect | | or |
| Ex. | Purification method | Intermediates | [M − H]⁻ |
| 2 | 6-(4-benzylpiperazine-1-carbonyl)- | Intermediate A-1 | 352.2 |
| | 4H-1,4-benzoxazin-3-one | and | |
| | Colorless solid | 1-benzylpiperazine | |
| | Preparative HPLC | (CAS RN 860027-50-5) | |
| 3 | 6-[4-[(4- | Intermediate A-1 | 369.2 |
| | fluorophenyl)methyl]piperidine-1- | and | |
| | carbonyl]-4H-1,4-benzoxazin-3-one | 4-(4- | |
| | Colorless solid | fluorobenzyl)piperidine | |
| | Preparative HPLC | (CAS RN 92822-02-1) | |
| 4 | 6-[4-[[4-(trifluoro- | Intermediate A-1 | 419.2 |
| | methyl)phenyl]methyl]piperidine-1- | and | |
| | carbonyl]-4H-1,4-benzoxazin-3-one | -(4-(trifluoro- | |
| | Colorless solid | methyl)benzyl)piperidine | |
| | Preparative HPLC | hydrochloride | |
| | | (CAS RN 192990-03-7) | |
| 5 | 6-[4-[(4- | Intermediate A-1 | 381.2 |
| | methoxyphenyl)methyl]piperidine-1- | and | |
| | carbonyl]-4H-1,4-benzoxazin-3-one | 4-(4-methoxy-benzyl)- | |
| | Colorless solid | piperidine | |
| | Preparative HPLC | (CAS RN 37581-26-3) | |
| 6 | 6-[4-[(3- | Intermediate A-1 | 381.2 |
| | methoxyphenyl)methyl]piperidine-1- | and | |
| | carbonyl]-4H-1,4-benzoxazin-3-one | 4-(3-methoxy-benzyl)- | |
| | Colorless solid | piperidine | |
| | Preparative HPLC | (CAS RN 150019-61-7) | |
| 7 | 6-(4-benzyl-4-hydroxypiperidine-1- | Intermediate A-1 | 367.2 |
| | carbonyl)-4H-1,4-benzoxazin-3-one | and | |
| | Colorless oil | 4-benzyl-4- | |
| | Preparative HPLC | hydroxypiperidine | |
| | | (CAS RN 51135-96-7) | |
| 8 | 6-[4-[(3- | Intermediate A-1 | 369.1 |
| | fluorophenyl)methyl]piperidine-1- | and | |
| | carbonyl]-4H-1,4-benzoxazin-3-one | 4-(3-fluoro-benzyl)- | |
| | Colorless oil | piperidine | |
| | Preparative HPLC | (CAS RN 202126-85-0) | |
| 9 | 6-[4-[(3,5- | Intermediate A-1 | 387.2 |
| | difluorophenyl)methyl]piperidine-1- | and | |
| | carbonyl]-4H-1,4-benzoxazin-3-one | 4-[(3,5- | |
| | Colorless solid | difluorophenyl)methyl] | |
| | Preparative HPLC | piperidine | |
| | | (CAS RN 94500-98-4) | |
| 10 | 6-[4-(benzenesulfonyl)piperidine-1- | Intermediate A-1 | 401.1 |
| | carbonyl]-4H-1,4-benzoxazin-3-one | and | |
| | Colorless solid | 4-(phenylsulfonyl)piperidine | |
| | Preparative HPLC | (CAS RN 285995-13-3) | |
| 11 | 6-[4-[(5S)-2-oxo-5-phenyl-1,3- | Intermediate A-1 | 422.2 |
| | oxazolidin-3-yl]piperidine-1- | and | |
| | carbonyl]- | Intermediate B-1 | |
| | 4H-1,4-benzoxazin-3-one | | |
| | Colorless solid | | |
| | Preparative HPLC | | |
| 12 | 6-[4-[(4- | Intermediate A-1 | 385.2 |
| | chlorophenyl)methyl]piperidine-1- | and | |
| | carbonyl]-4H-1,4-benzoxazin-3-one | 4-(4-Chlorobenzyl)piperidine | |
| | Colorless solid | (CAS RN 36938-76-8) | |
| | Preparative HPLC | | |
| 13 | 6-(4-benzoylpiperidine-1-carbonyl)- | Intermediate A-1 | 365.3 |
| | 4H-1,4-benzoxazin-3-one | and | |
| | Colorless solid | 4-benzoylpiperidine | |
| | Preparative HPLC | (CAS RN 37586-22-4) | |
| 14 | 6-(4-phenoxypiperidine-1-carbonyl)- | Intermediate A-1 | 353.2 |
| | 4H-1,4-benzoxazin-3-one | and | |
| | Colorless solid | 4-phenoxy-piperidine | |
| | Preparative HPLC | (CAS RN 3202-33-3) | |
| 15 | 6-[4-[4- | Intermediate A-1 | 421.3 |
| | (trifluoromethyl)phenoxy]piperidine- | and | |
| | 1-carbonyl]-4H-1,4-benzoxazin-3-one | 4-(4-(trifluoro- | |
| | Colorless solid | methyl)phenoxy)piperidine | |
| | Preparative HPLC | (CAS RN 28033-37-6) | |
| 16 | 6-[4-[(2,3- | Intermediate A-1 | 387.2 |
| | difluorophenyl)methyl]piperidine-1- | and | |
| | carbonyl]-4H-1,4-benzoxazin-3-one | 4-[(2,3- | |
| | Colorless solid | difluorophenyl)methyl] | |
| | Preparative HPLC | piperidine | |
| | | (CAS RN 253450-32-7) | |
| 17 | 6-[4-[[3-(trifluoro- | Intermediate A-1 | 419.2 |
| | methyl)phenyl(methyl(piperidine-1- | and | |
| | carbonyl]-4H-1,4-benzoxazin-3-one | 4-(3-(trifluoro- | |
| | Colorless solid | methyl)benzyl)piperidine | |
| | Preparative HPLC | (CAS RN 37581-28-5) | |
| 18 | 6-(3-phenoxypyrrolidine-1-carbonyl)- | Intermediate A-1 | 339.1 |
| | 4H-1,4-benzoxazin-3-one | and | |
| | Off white solid | 3-phenoxypyrrolidine | |
| | Flash chromatography | (CAS RN 931581-76-9) | |
| 19 | 6-[4-[(5R)-2-oxo-5-phenyl-1,3- | Intermediate A-1 | 422.4 |
| | oxazolidin-3-yl]piperidine-1- | and | |
| | carbonyl]-4H-1,4-benzoxazin-3-one | Intermediate B-2 | |
| | Light brown solid | | |
| | Flash chromatography | | |
| 20 | 4-[[1-(3-oxo-4H-1,4-benzoxazine-6- | Intermediate A-1 | 376.3 |
| | carbonyl)piperidin-4- | and | |
| | yl]methyl]benzonitrile | 4-(4- | |
| | Colorless solid | (cyanobenzyl)piperidine | |
| | Preparative HPLC | (CAS RN 333987-57-8) | |
| 21 | 6-[4-[(2- | Intermediate A-1 | 427.3 |
| | phenylphenyl)methyl]piperidine-1- | and | |
| | carbonyl]-4H-1,4-benzoxazin-3-one | 4-([1,1′-biphenyl]-2- | |
| | Colorless solid | ylmethyl)piperidine | |
| | Flash chromatography | (CAS RN 1800372-42-2) | |
| 22 | 6-[4-(pyridin-2-ylmethyl)piperidine-1- | Intermediate A-1 | 352.3 |
| | carbonyl]-4H-1,4-benzoxazin-3-one | and | |
| | Colorless solid | 2-(piperidin-4- | |
| | Preparative HPLC | ylmethyl)pyridine | |
| | | (CAS RN 811812-57-4) | |
| 23 | 6-[4-[(4-chlorophenyl)methyl]-4- | Intermediate A-1 | 403.2 |
| | fluoropiperidine-1-carbonyl]-4H-1,4- | and | |
| | benzoxazin-3-one | 4-[(4- | |
| | Colorless solid | chlorophenyl)methyl]- | |
| | Flash chromatography | 4-fluoropiperidine | |
| | | (CAS RN 1564467-17-9) | |
| 24 | 6-(4-benzylpiperidine-1-carbonyl)-8- | Intermediate A-2 | 369.3 |
| | fluoro-4H-1,4-benzoxazin-3-one | and | |
| | Colorless solid | 4-benzylpiperidine | |
| | Preparative HPLC | (CAS RN 31252-42-3) | |
| 25 | N-methyl-N-[1-(3-oxo-4H-1,4- | Intermediate A-1 | 394.2 |
| | benzoxazine-6-carbonyl)piperidin-4- | and | |
| | yl]benzamide | N-methyl-n-(piperidin- | |
| | Colorless solid | 4-yl)benzamide | |
| | Preparative HPLC | (CAS RN 64951-39-9) | |
| 26 | 6-(4-benzylpiperidine-1-carbonyl)-4H- | 3-oxo-3,4-dihydro-2H- | 352.2 |
| | pyrido[3,2-b][1,4]oxazin-3-one | pyrido[3,2- | |
| | Ligth yellow foam | b][1,4]oxazine-6- | |
| | Flash chromatography | carboxylic acid | |
| | | (CAS RN 337463-89-5) | |
| | | and | |
| | | 4-benzylpiperidine | |
| | | (CAS RN 31252-42-3) | |
| 27 | 6-[4-(piperidine-1- | Intermediate A-1 | 372.2 |
| | carbonyl)piperidine-1-carbonyl]-4H- | and | |
| | 1,4-benzoxazin-3-one | 1-(piperidin-4- | |
| | Colorless solid | ylcarbonyl)piperidine | |
| | Flash chromatography | (CAS RN 63214-58-4) | |
| 28 | 6-(3-benzylpyrrolidine-1-carbonyl)- | Intermediate A-1 | 337.1 |
| | 4H-1,4-benzoxazin-3-one | and | |
| | Colorless foam | 3-benzylpyrrolidine | |
| | Flash chromatography | (CAS RN 170304-83-3) | |
| 29 | 6-[3-[[4-(trifluoro- | Intermediate A-1 | 391.1 |
| | methyl)phenyl]methyl]azetidine-1- | and | |
| | carbonyl]-4H-1,4-benzoxazin-3-one | 3-[[4-(trifluoro- | |
| | Light yellow amorphous | methyl)phenyl]methyl]azetidine | |
| | Flash chromatography | (CAS RN 937614-88-5) | |
| 30 | 6-[3-[[3-(trifluoro- | Intermediate A-1 | 405.1 |
| | methyl)phenyl]methyl]pyrrolidine-1- | and | |
| | carbonyl]-4H-1,4-benzoxazin-3-one | 3-(trifluoromethyl)benzyl) | |
| | Light yellow amorphous | pyrrolidine | |
| | Flash chromatography | (CAS RN 1158757-88-0) | |
| 31 | 6-[3-[[4-(trifluoro- | Intermediate A-1 | 405.1 |
| | methyl)phenyl]methyl]pyrrolidine-1- | and | |
| | carbonyl]-4H-1,4-benzoxazin-3-one | 4-(Trifluoromethyl)benzyl) | |
| | Colorless solid | pyrrolidine | |
| | Flash chromatography | (CAS RN 957998-84-4) | |
| 32 | 6-[4-[4-(trifluoro- | Intermediate A-1 | 434.1 |
| | methyl)benzoyl]piperazine-1- | and | |
| | carbonyl]-4H-1,4-benzoxazin-3-one | 1-[4-(trifluoro- | |
| | Light yellow solid | methyl)benzoyl]piperazine | |
| | Flash chromatography | (CAS RN 179334-12-4) | |
| 33 | N-cyclopropyl-N-[1-(3-oxo-4H-1,4- | Intermediate A-1 | 434.3 |
| | benzoxazine-6-carbonyl)piperidin-4- | and | |
| | yl]-2-phenylacetamide | N-benzyl-N- | |
| | Colorless solid | cyclopropylpiperidine- | |
| | Flash chromatography | 4-carboxamide | |
| | | (CAS RN 1154254-69-9) | |

### Intermediate C-1

### 6-(3-(1H-Pyrazol-3-yl)piperazine-1-carbonyl)-2H-benzo[b][1,4]oxazin-3(4H)-one

3-Oxo-3,4-dihydro-2H-benzo[b][1,4]oxazine-6-carboxylic acid (50 mg, 259 μmol, CAS RN 134997-87-8), 2-(1H-pyrazol-3-yl)piperazine (43.3 mg, 285 μmol, CAS RN 111781-55-6), HATU (118 mg, 311 μmol) and TEA (78.6 mg, 108 μL, 777 μmol) were dissolved in DMF (1.23 mL). The reaction mixture was stirred at RT for 48 hr. The reaction mixture was concentrated in vacuo. The residue was dissolved with MeOH then treated with silica gel. The suspension was concentrated and the residue was purified by silica gel chromatography (12 g silica gel column, 0-100% EtOAc in n-heptane) to give a yellow solid (83 mg, 98%); MS(ESI): m/z=328.3 [M+H]⁺

### Intermediate C-2

### 6-(3-(Trifluoromethyl)piperazine-1-carbonyl)-2H-benzo[b][1,4]oxazin-3(4H)-one

3-Oxo-3,4-dihydro-2H-benzo[b][1,4]oxazine-6-carboxylic acid (100 mg, 518 μmol, CAS RN 134997-87-8), 2-(trifluoromethyl)piperazine (87.8 mg, 569 μmol, CAS RN 131922-05-9), HATU (236 mg, 621 μmol) and TEA (157 mg, 216 μl, 1.55 mmol) were dissolved in DMF (2.5 mL). The reaction mixture was stirred at RT for 15 hr. The reaction mixture was purified by prep. HPLC to give an off-white solid (144 mg, 82.7%); MS(ESI): m/z=330.2 [M+H]⁺.

### Intermediate C-3

### 6-(3-Methylpiperazine-1-carbonyl)-2H-benzo[b][1,4]oxazin-3(4H)-one

3-Oxo-3,4-dihydro-2H-benzo[b][1,4]oxazine-6-carboxylic acid (100 mg, 518 μmol, CAS RN 134997-87-8), 2-methylpiperazine (57 mg, 569 μmol, CAS RN-109-07-9), HATU (236 mg. 621 μmol) and TEA (157 mg, 216 μl, 1.55 mmol) were dissolved in DMF (2.5 ml). The reaction solution was stirred at RT for 15 h. The reaction mixture was purified by prep. HPLC to give a yellow solid (123 mg, 86.3%); MS(ESI): m/z=276.2 [M+H]⁺

### Example 34

### 6-[3-Methyl-4-[[4-(trifluoromethyl) phenyl]methyl]piperazine-1-carbonyl]-4H-1,4-benzoxazin-3-one

6-(3-Methylpiperazine-1-carbonyl)-2H-benzo[b][1,4]oxazin-3(4H)-one (116 mg, 421 μmol, intermediate C-3), 4-(trifluoromethyl)benzaldehyde (88 mg, 67.6 μl, 506 μmol, CAS RN 455-19-6) and sodium triacetoxyborohydride (89.3 mg, 421 μmol, CAS RN 56553-60-7) in DCM (1.7 mL) were stirred at RT for 72 h. The reaction mixture was concentrated. The residue was purified by flash chromatography (24 g silica gel column, 0-10% MeOH in DCM) to give a white solid (12 mg. 6.57%): MS(ESI): m/z=434.3 [M+H]⁺.

The following examples listed in Table 3 were prepared in analogy to the procedures described for the preparation of example 34 by using the indicated intermediates and/or commercial compounds and using the mentioned purification method such as preparative HPLC (Gemini NX column) or silica gel flash chromatography.

TABLE 3

| | | | MS, m/z |
|---|---|---|---|
| | Name | | [M + H]⁺ |
| | Aspect | | or |
| Ex. | Purification method | Intermediates | [M − H]⁻ |
| 35 | 6-[4-[2-(3-chlorophenyl)ethyl]-3- | Intermediate C-1 | 466.3 |
| | (1H-pyrazol-3-yl)piperazine-1- | and | |
| | carbonyl]-4H-1,4-benzoxazin-3- | 2-(3-chlorophenyl) | |
| | one | acetaldehyde | |
| | White solid | (CAS RN | |
| | Preparative HPLC | 41904-40-9) | |
| 36 | 6-[3-(trifluoromethyl)-4-[[4- | Intermediate C-2 | 487.1 |
| | (trifluoro- | and | |
| | methyl)phenyl]methyl]piperazine- | 4-(trifluoromethyl) | |
| | 1-carbonyl]-4H-1,4-benzoxazin-3- | benzaldehyde | |
| | one | (CAS RN | |
| | White solid | 455-19-6) | |
| | Preparative HPLC | | |
| 37 | 6-[3-(1H-pyrazol-3-yl)-4-[[4- | Intermediate C-1 | 486.4 |
| | (trifluoro- | and | |
| | methyl)phenyl]methyl]piperazine- | 4-(trifluoromethyl) | |
| | 1-carbonyl]-4H-1,4-benzoxazin-3- | benzaldehyde | |
| | one | (CAS RN | |
| | White solid | 455-19-6) | |
| | Preparative HPLC | | |

### Example A

A compound of formula (I) can be used in a manner known per se as the active ingredient for the production of tablets of the following composition:

| | Per tablet | |
|---|---|---|
| Active ingredient | 200 | mg |
| Microcrystalline cellulose | 155 | mg |
| Corn starch | 25 | mg |
| Talc | 25 | mg |
| Hydroxypropylmethylcellulose | 20 | mg |
| | 425 | mg |

### Example B

A compound of formula (I) can be used in a manner known per se as the active ingredient for the production of capsules of the following composition:

| | Per capsule | |
|---|---|---|
| Active ingredient | 100.0 | mg |
| Corn starch | 20.0 | mg |
| Lactose | 95.0 | mg |
| Talc | 4.5 | mg |
| Magnesium stearate | 0.5 | mg |
| | 220.0 | mg |

1.-36. (canceled)

37. A method of inhibiting monoacylglycerol lipase in a human, comprising administering an effective amount of a compound to the human thereby inhibiting monoacylglycerol lipase, wherein the compound is a compound of formula (I): 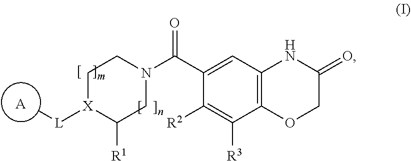
or a pharmaceutically acceptable salt thereof, wherein:
A is selected from the group consisting of aryl, heteroaryl, cycloalkyl, and heterocyclyl, wherein each of said aryl, heteroaryl, cycloalkyl, and heterocyclyl is independently substituted with R⁴ and R⁵;
L is selected from the group consisting of heterocyclyl, —O—, —C(O)—, —S(O)₂—, —CHR⁶—, —CH₂CH₂—, —(CH₂)ₚ—C(O)—NR⁷—, and —(CH₂)_{q}—NR⁸—C(O)—;
X is N or C—R⁹,
each of m, n, p, and q is independently an integer selected from the group consisting of 0 and 1; and
each of R¹, R², R³, R⁴, R⁵, R⁶, R⁷, R⁸, and R⁹ is independently selected from the group consisting of hydrogen, hydroxy, halogen, cyano, alkyl, haloalkyl, cycloalkyl, alkoxy, haloalkoxy, aryl, and heteroaryl.

38. The method of claim 37, wherein:
A is selected from the group consisting of aryl, heteroaryl, and heterocyclyl, wherein each of said aryl, heteroaryl, and heterocyclyl is independently substituted with R⁴ and R⁵;
R⁴ is selected from the group consisting of hydrogen, halogen, haloalkyl, alkoxy, cyano, and aryl; and
R⁵ is hydrogen or halogen.

39. The method of claim 37, wherein:
A is aryl substituted with R⁴ and R⁵;
R⁴ is selected from the group consisting of hydrogen, halogen, haloalkyl, and alkoxy; and
R⁵ is hydrogen or halogen.

40. The method of claim 37, wherein:
A is phenyl substituted with R⁴ and R⁵;
R⁴ is selected from the group consisting of hydrogen, fluorine, chlorine, trifluoromethyl, and methoxy; and
R⁵ is hydrogen or fluorine.

41. The method of claim 37, wherein:
L is selected from the group consisting of heterocyclyl, —O—, —C(O)—, —S(O)₂—, —CH₂—, —CH₂CH₂—, and —(CH₂)ₚ—C(O)—NR⁷—;
p is 0 or 1; and
R⁷ is alkyl or cycloalkyl.

42. The method of claim 37, wherein:
A is selected from the group consisting of aryl, heteroaryl, and heterocyclyl, wherein each of said aryl, heteroaryl, and heterocyclyl is independently substituted with R⁴ and R⁵;
L is selected from the group consisting of heterocyclyl, —O—, —C(O)—, —S(O)₂—, —CH₂—, —CH₂CH₂—, and —(CH₂)ₚ—C(O)—NR⁷—;
X is N or C—R⁹,
each of m, n, and p is independently an integer selected from the group consisting of 0 and 1;
R¹ is selected from the group consisting of hydrogen, alkyl, haloalkyl, and heteroaryl;
R² and R³ are both hydrogen;
R⁴ is selected from the group consisting of hydrogen, halogen, haloalkyl, alkoxy, cyano and aryl;
R⁵ is hydrogen or halogen;
R⁷ is alkyl or cycloalkyl; and
R⁹ is selected from the group consisting of hydrogen, hydroxy and halogen.

43. The method of claim 37, wherein:
A is aryl substituted with R⁴ and R⁵;
L is —O— or —CH₂—;
X is C—H,
m and n are both 1;
each of R¹, R², and R³ is hydrogen;
R⁴ is selected from the group consisting of hydrogen, halogen, haloalkyl, and alkoxy; and
R⁵ is hydrogen or halogen.

44. The method of claim 37, wherein:
A is phenyl substituted with R⁴ and R⁵;
L is —O— or —CH₂—;
X is C—H,
m and n are both 1;
each of R¹, R², and R³ is hydrogen;
R⁴ is selected from the group consisting of hydrogen, fluorine, chlorine, trifluoromethyl, and methoxy; and
R⁵ is hydrogen or fluorine.

45. The method of claim 37, wherein the compound is selected from the group consisting of:
6-(4-benzylpiperidine-1-carbonyl)-4H-1,4-benzoxazin-3-one;
6-(4-benzylpiperazine-1-carbonyl)-4H-1,4-benzoxazin-3-one;
6-[4-[(4-fluorophenyl)methyl]piperidine-1-carbonyl]-4H-1,4-benzoxazin-3-one;
6-[4-[[4-(trifluoromethyl)phenyl]methyl]piperidine-1-carbonyl]-4H-1,4-benzoxazin-3-one;
6-[4-[(4-methoxyphenyl)methyl]piperidine-1-carbonyl]-4H-1,4-benzoxazin-3-one;
6-[4-[(3-methoxyphenyl)methyl]piperidine-1-carbonyl]-4H-1,4-benzoxazin-3-one;
6-(4-benzyl-4-hydroxypiperidine-1-carbonyl)-4H-1,4-benzoxazin-3-one;
6-[4-[(3-fluorophenyl)methyl]piperidine-1-carbonyl]-4H-1,4-benzoxazin-3-one;
6-[4-[(3,5-difluorophenyl)methyl]piperidine-1-carbonyl]-4H-1,4-benzoxazin-3-one;
6-[4-(benzenesulfonyl)piperidine-1-carbonyl]-4H-1,4-benzoxazin-3-one;
6-[4-[(5S)-2-oxo-5-phenyl-1,3-oxazolidin-3-yl]piperidine-1-carbonyl]-4H-1,4-benzoxazin-3-one;
6-[4-[(4-chlorophenyl)methyl]piperidine-1-carbonyl]-4H-1,4-benzoxazin-3-one;
6-(4-benzoylpiperidine-1-carbonyl)-4H-1,4-benzoxazin-3-one;
6-(4-phenoxypiperidine-1-carbonyl)-4H-1,4-benzoxazin-3-one;
6-[4-[4-(trifluoromethyl)phenoxy]piperidine-1-carbonyl]-4H-1,4-benzoxazin-3-one;
6-[4-[(2,3-difluorophenyl)methyl]piperidine-1-carbonyl]-4H-1,4-benzoxazin-3-one;
6-[4-[[3-(trifluoromethyl)phenyl]methyl]piperidine-1-carbonyl]-4H-1,4-benzoxazin-3-one;
6-(3-phenoxypyrrolidine-1-carbonyl)-4H-1,4-benzoxazin-3-one;
6-[4-[(5R)-2-oxo-5-phenyl-1,3-oxazolidin-3-yl]piperidine-1-carbonyl]-4H-1,4-benzoxazin-3-one;
4-[[1-(3-oxo-4H-1,4-benzoxazine-6-carbonyl)piperidin-4-yl]methyl]benzonitrile;
6-[4-[(2-phenylphenyl)methyl]piperidine-1-carbonyl]-4H-1,4-benzoxazin-3-one;
6-[4-(pyridin-2-ylmethyl)piperidine-1-carbonyl]-4H-1,4-benzoxazin-3-one;
6-[4-[(4-chlorophenyl)methyl]-4-fluoropiperidine-1-carbonyl]-4H-1,4-benzoxazin-3-one;
6-(4-benzylpiperidine-1-carbonyl)-8-fluoro-4H-1,4-benzoxazin-3-one;
N-methyl-N-[1-(3-oxo-4H-1,4-benzoxazine-6-carbonyl)piperidin-4-yl]benzamide;
6-(4-benzylpiperidine-1-carbonyl)-4H-pyrido[3,2-b][1,4]oxazin-3-one;
6-[4-(piperidine-1-carbonyl)piperidine-1-carbonyl]-4H-1,4-benzoxazin-3-one;
6-(3-benzylpyrrolidine-1-carbonyl)-4H-1,4-benzoxazin-3-one;
6-[3-[[4-(trifluoromethyl)phenyl]methyl]azetidine-1-carbonyl]-4H-1,4-benzoxazin-3-one;
6-[3-[[3-(trifluoromethyl)phenyl]methyl]pyrrolidine-1-carbonyl]-4H-1,4-benzoxazin-3-one;
6-[3-[[4-(trifluoromethyl)phenyl]methyl]pyrrolidine-1-carbonyl]-4H-1,4-benzoxazin-3-one;
6-[4-[4-(trifluoromethyl)benzoyl]piperazine-1-carbonyl]-4H-1,4-benzoxazin-3-one;
N-cyclopropyl-N-[1-(3-oxo-4H-1,4-benzoxazine-6-carbonyl)piperidin-4-yl]-2-phenylacetamide;
6-[4-[2-(3-chlorophenyl)ethyl]-3-(1H-pyrazol-3-yl)piperazine-1-carbonyl]-4H-1,4-benzoxazin-3-one;
6-[3-(trifluoromethyl)-4-[[4-(trifluoromethyl)phenyl]methyl]piperazine-1-carbonyl]-4H-1,4-benzoxazin-3-one;
6-[3-(1H-pyrazol-3-yl)-4-[[4-(trifluoromethyl)phenyl]methyl]piperazine-1-carbonyl]-4H-1,4-benzoxazin-3-one; and
6-[3-methyl-4-[[4-(trifluoromethyl)phenyl]methyl]piperazine-1-carbonyl]-4H-1,4-benzoxazin-3-one;
or is a pharmaceutically acceptable salt thereof.

46. The method of claim 37, wherein the compound is selected from the group consisting of:
6-(4-benzylpiperidine-1-carbonyl)-4H-1,4-benzoxazin-3-one;
6-[4-[(4-fluorophenyl)methyl]piperidine-1-carbonyl]-4H-1,4-benzoxazin-3-one;
6-[4-[[4-(trifluoromethyl)phenyl]methyl]piperidine-1-carbonyl]-4H-1,4-benzoxazin-3-one;
6-[4-[(3-methoxyphenyl)methyl]piperidine-1-carbonyl]-4H-1,4-benzoxazin-3-one;
6-[4-[(3-fluorophenyl)methyl]piperidine-1-carbonyl]-4H-1,4-benzoxazin-3-one;
6-[4-[(3,5-difluorophenyl)methyl]piperidine-1-carbonyl]-4H-1,4-benzoxazin-3-one;
6-[4-[(4-chlorophenyl)methyl]piperidine-1-carbonyl]-4H-1,4-benzoxazin-3-one;
6-[4-[4-(trifluoromethyl)phenoxy]piperidine-1-carbonyl]-4H-1,4-benzoxazin-3-one;
6-[4-[(2,3-difluorophenyl)methyl]piperidine-1-carbonyl]-4H-1,4-benzoxazin-3-one; and
6-[4-[[3-(trifluoromethyl)phenyl]methyl]piperidine-1-carbonyl]-4H-1,4-benzoxazin-3-one;
or a pharmaceutically acceptable salt thereof.

47. A method of treating multiple sclerosis, Alzheimer's disease, Parkinson's disease, amyotrophic lateral sclerosis, traumatic brain injury, neurotoxicity, stroke, epilepsy, anxiety, migraine, depression, cancer, or pain in a human, comprising administering an effective amount of a compound to the human thereby treating the multiple sclerosis, Alzheimer's disease, Parkinson's disease, amyotrophic lateral sclerosis, traumatic brain injury, neurotoxicity, stroke, epilepsy, anxiety, migraine, depression, cancer, or pain, wherein the compound is a compound of formula (I): 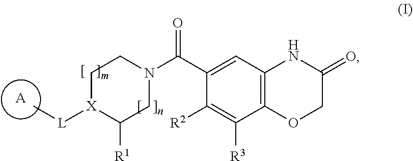
or a pharmaceutically acceptable salt thereof, wherein:
A is selected from the group consisting of aryl, heteroaryl, cycloalkyl, and heterocyclyl, wherein each of said aryl, heteroaryl, cycloalkyl, and heterocyclyl is independently substituted with R⁴ and R⁵;
L is selected from the group consisting of heterocyclyl, —O—, —C(O)—, —S(O)₂—, —CHR⁶—, —CH₂CH₂—, —(CH₂)ₚ—C(O)—NR⁷—, and —(CH₂)_{q}—NR⁸—C(O)—;
X is N or C—R⁹,
each of m, n, p, and q is independently an integer selected from the group consisting of 0 and 1; and
each of R¹, R², R³, R⁴, R⁵, R⁶, R⁷, R⁸, and R⁹ is independently selected from the group consisting of hydrogen, hydroxy, halogen, cyano, alkyl, haloalkyl, cycloalkyl, alkoxy, haloalkoxy, aryl, and heteroaryl.

48. The method of claim 47, wherein:
A is selected from the group consisting of aryl, heteroaryl, and heterocyclyl, wherein each of said aryl, heteroaryl, and heterocyclyl is independently substituted with R⁴ and R⁵;
R⁴ is selected from the group consisting of hydrogen, halogen, haloalkyl, alkoxy, cyano, and aryl; and
R⁵ is hydrogen or halogen.

49. The method of claim 47, wherein:
A is aryl substituted with R⁴ and R⁵;
R⁴ is selected from the group consisting of hydrogen, halogen, haloalkyl, and alkoxy; and
R⁵ is hydrogen or halogen.

50. The method of claim 47, wherein:
A is phenyl substituted with R⁴ and R⁵;
R⁴ is selected from the group consisting of hydrogen, fluorine, chlorine, trifluoromethyl, and methoxy; and
R⁵ is hydrogen or fluorine.

51. The method of claim 47, wherein:
L is selected from the group consisting of heterocyclyl, —O—, —C(O)—, —S(O)₂—, —CH₂—, —CH₂CH₂—, and —(CH₂)ₚ—C(O)—NR⁷—;
p is 0 or 1; and
R⁷ is alkyl or cycloalkyl.

52. The method of claim 47, wherein:
A is selected from the group consisting of aryl, heteroaryl, and heterocyclyl, wherein each of said aryl, heteroaryl, and heterocyclyl is independently substituted with R⁴ and R⁵;
L is selected from the group consisting of heterocyclyl, —O—, —C(O)—, —S(O)₂—, —CH₂—, —CH₂CH₂—, and —(CH₂)ₚ—C(O)—NR⁷—;
X is N or C—R⁹,
each of m, n, and p is independently an integer selected from the group consisting of 0 and 1;
R¹ is selected from the group consisting of hydrogen, alkyl, haloalkyl, and heteroaryl;
R² and R³ are both hydrogen;
R⁴ is selected from the group consisting of hydrogen, halogen, haloalkyl, alkoxy, cyano and aryl;
R⁵ is hydrogen or halogen;
R⁷ is alkyl or cycloalkyl; and
R⁹ is selected from the group consisting of hydrogen, hydroxy and halogen.

53. The method of claim 47, wherein:
A is aryl substituted with R⁴ and R⁵;
L is —O— or —CH₂—;
X is C—H,
m and n are both 1;
each of R¹, R², and R³ is hydrogen;
R⁴ is selected from the group consisting of hydrogen, halogen, haloalkyl, and alkoxy; and
R⁵ is hydrogen or halogen.

54. The method of claim 47, wherein:
A is phenyl substituted with R⁴ and R⁵;
L is —O— or —CH₂—;
X is C—H,
m and n are both 1;
each of R¹, R², and R³ is hydrogen;
R⁴ is selected from the group consisting of hydrogen, fluorine, chlorine, trifluoromethyl, and methoxy; and
R⁵ is hydrogen or fluorine.

55. The method of claim 47, wherein the compound is selected from the group consisting of:
6-(4-benzylpiperidine-1-carbonyl)-4H-1,4-benzoxazin-3-one;
6-(4-benzylpiperazine-1-carbonyl)-4H-1,4-benzoxazin-3-one;
6-[4-[(4-fluorophenyl)methyl]piperidine-1-carbonyl]-4H-1,4-benzoxazin-3-one;
6-[4-[[4-(trifluoromethyl)phenyl]methyl]piperidine-1-carbonyl]-4H-1,4-benzoxazin-3-one;
6-[4-[(4-methoxyphenyl)methyl]piperidine-1-carbonyl]-4H-1,4-benzoxazin-3-one;
6-[4-[(3-methoxyphenyl)methyl]piperidine-1-carbonyl]-4H-1,4-benzoxazin-3-one;
6-(4-benzyl-4-hydroxypiperidine-1-carbonyl)-4H-1,4-benzoxazin-3-one;
6-[4-[(3-fluorophenyl)methyl]piperidine-1-carbonyl]-4H-1,4-benzoxazin-3-one;
6-[4-[(3,5-difluorophenyl)methyl]piperidine-1-carbonyl]-4H-1,4-benzoxazin-3-one;
6-[4-(benzenesulfonyl)piperidine-1-carbonyl]-4H-1,4-benzoxazin-3-one;
6-[4-[(5S)-2-oxo-5-phenyl-1,3-oxazolidin-3-yl]piperidine-1-carbonyl]-4H-1,4-benzoxazin-3-one;
6-[4-[(4-chlorophenyl)methyl]piperidine-1-carbonyl]-4H-1,4-benzoxazin-3-one;
6-(4-benzoylpiperidine-1-carbonyl)-4H-1,4-benzoxazin-3-one;
6-(4-phenoxypiperidine-1-carbonyl)-4H-1,4-benzoxazin-3-one;
6-[4-[4-(trifluoromethyl)phenoxy]piperidine-1-carbonyl]-4H-1,4-benzoxazin-3-one;
6-[4-[(2,3-difluorophenyl)methyl]piperidine-1-carbonyl]-4H-1,4-benzoxazin-3-one;
6-[4-[[3-(trifluoromethyl)phenyl]methyl]piperidine-1-carbonyl]-4H-1,4-benzoxazin-3-one;
6-(3-phenoxypyrrolidine-1-carbonyl)-4H-1,4-benzoxazin-3-one;
6-[4-[(5R)-2-oxo-5-phenyl-1,3-oxazolidin-3-yl]piperidine-1-carbonyl]-4H-1,4-benzoxazin-3-one;
4-[[1-(3-oxo-4H-1,4-benzoxazine-6-carbonyl)piperidin-4-yl]methyl]benzonitrile;
6-[4-[(2-phenylphenyl)methyl]piperidine-1-carbonyl]-4H-1,4-benzoxazin-3-one;
6-[4-(pyridin-2-ylmethyl)piperidine-1-carbonyl]-4H-1,4-benzoxazin-3-one;
6-[4-[(4-chlorophenyl)methyl]-4-fluoropiperidine-1-carbonyl]-4H-1,4-benzoxazin-3-one;
6-(4-benzylpiperidine-1-carbonyl)-8-fluoro-4H-1,4-benzoxazin-3-one;
N-methyl-N-[1-(3-oxo-4H-1,4-benzoxazine-6-carbonyl)piperidin-4-yl]benzamide;
6-(4-benzylpiperidine-1-carbonyl)-4H-pyrido[3,2-b][1,4]oxazin-3-one;
6-[4-(piperidine-1-carbonyl)piperidine-1-carbonyl]-4H-1,4-benzoxazin-3-one;
6-(3-benzylpyrrolidine-1-carbonyl)-4H-1,4-benzoxazin-3-one;
6-[3-[[4-(trifluoromethyl)phenyl]methyl]azetidine-1-carbonyl]-4H-1,4-benzoxazin-3-one;
6-[3-[[3-(trifluoromethyl)phenyl]methyl]pyrrolidine-1-carbonyl]-4H-1,4-benzoxazin-3-one;
6-[3-[[4-(trifluoromethyl)phenyl]methyl]pyrrolidine-1-carbonyl]-4H-1,4-benzoxazin-3-one;
6-[4-[4-(trifluoromethyl)benzoyl]piperazine-1-carbonyl]-4H-1,4-benzoxazin-3-one;
N-cyclopropyl-N-[1-(3-oxo-4H-1,4-benzoxazine-6-carbonyl)piperidin-4-yl]-2-phenylacetamide;
6-[4-[2-(3-chlorophenyl)ethyl]-3-(1H-pyrazol-3-yl)piperazine-1-carbonyl]-4H-1,4-benzoxazin-3-one;
6-[3-(trifluoromethyl)-4-[[4-(trifluoromethyl)phenyl]methyl]piperazine-1-carbonyl]-4H-1,4-benzoxazin-3-one;
6-[3-(1H-pyrazol-3-yl)-4-[[4-(trifluoromethyl)phenyl]methyl]piperazine-1-carbonyl]-4H-1,4-benzoxazin-3-one; and
6-[3-methyl-4-[[4-(trifluoromethyl)phenyl]methyl]piperazine-1-carbonyl]-4H-1,4-benzoxazin-3-one;
or is a pharmaceutically acceptable salt thereof.

56. The method of claim 47, wherein the compound is selected from the group consisting of:
6-(4-benzylpiperidine-1-carbonyl)-4H-1,4-benzoxazin-3-one;
6-[4-[(4-fluorophenyl)methyl]piperidine-1-carbonyl]-4H-1,4-benzoxazin-3-one;
6-[4-[[4-(trifluoromethyl)phenyl]methyl]piperidine-1-carbonyl]-4H-1,4-benzoxazin-3-one;
6-[4-[(3-methoxyphenyl)methyl]piperidine-1-carbonyl]-4H-1,4-benzoxazin-3-one;
6-[4-[(3-fluorophenyl)methyl]piperidine-1-carbonyl]-4H-1,4-benzoxazin-3-one;
6-[4-[(3,5-difluorophenyl)methyl]piperidine-1-carbonyl]-4H-1,4-benzoxazin-3-one;
6-[4-[(4-chlorophenyl)methyl]piperidine-1-carbonyl]-4H-1,4-benzoxazin-3-one;
6-[4-[4-(trifluoromethyl)phenoxy]piperidine-1-carbonyl]-4H-1,4-benzoxazin-3-one;
6-[4-[(2,3-difluorophenyl)methyl]piperidine-1-carbonyl]-4H-1,4-benzoxazin-3-one; and
6-[4-[[3-(trifluoromethyl)phenyl]methyl]piperidine-1-carbonyl]-4H-1,4-benzoxazin-3-one;
or a pharmaceutically acceptable salt thereof.
